(19) United States
(12) Patent Application Publication
(10) Pub. No.: US 2006/0088728 A1
(43) Pub. Date: Apr. 27, 2006

(54) Arylcarbazoles as hosts in PHOLEDs

(76) Inventors: Raymond Kwong, Plainsboro, NJ (US); David Knowles, Apollo, PA (US); William Ceyrolles, Coraopolis, PA (US); Bin Ma, Monroeville, PA (US)

Correspondence Address: KENYON & KENYON LLP, ONE BROADWAY, NEW YORK, NY, 10004, US

(21) Appl. No.: 10/971,844

(22) Filed: Oct. 22, 2004

(51) Int. Cl. H01L 51/54; H05B 33/14; C09K 11/06; C07D 209/82

(52) U.S. Cl. 428/690; 428/917; 313/504; 313/506; 548/440; 257/40; 257/102; 257/103; 257/E51.026

(57) ABSTRACT

An organic light emitting device is provided. The device has an anode, a cathode and an emissive layer disposed between the anode and the cathode. The emissive layer includes a host material and a dopant, and the host material is selected from the group consisting of: 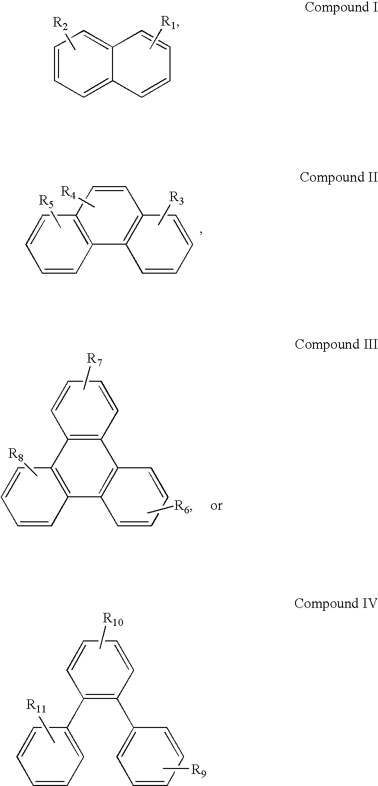 wherein each R represent no substitution, mono-, di-, or tri-substitution, and the substituents are the same or different, and may be alkyl, alkenyl, alkynyl, aryl, thioalkoxy, halo, haloalkyl, cyano, carbonyl, carboxyl, heteroaryl, and substituted aryl, and at least one R for each Compounds I, II, III, or IV includes a carbazole group.

### FIELD OF THE INVENTION

The present invention relates to organic light emitting devices (OLEDs), and specifically to phosphorescent organic materials used in such devices. More specifically, the present invention relates to arylcarbazole complexes incorporated into OLEDs.

### BACKGROUND

Opto-electronic devices that make use of organic materials are becoming increasingly desirable for a number of reasons. Many of the materials used to make such devices are relatively inexpensive, so organic opto-electronic devices have the potential for cost advantages over inorganic devices. In addition, the inherent properties of organic materials, such as their flexibility, may make them well suited for particular applications such as fabrication on a flexible substrate. Examples of organic opto-electronic devices include organic light emitting devices (OLEDs), organic phototransistors, organic photovoltaic cells, and organic photodetectors. For OLEDs, the organic materials may have performance advantages over conventional materials. For example, the wavelength at which an organic emissive layer emits light may generally be readily tuned with appropriate dopants.

As used herein, the term “organic” includes polymeric materials as well as small molecule organic materials that may be used to fabricate organic opto-electronic devices. “Small molecule” refers to any organic material that is not a polymer, and “small molecules” may actually be quite large. Small molecules may include repeat units in some circumstances. For example, using a long chain alkyl group as a substituent does not remove a molecule from the “small molecule” class. Small molecules may also be incorporated into polymers, for example as a pendent group on a polymer backbone or as a part of the backbone. Small molecules may also serve as the core moiety of a dendrimer, which consists of a series of chemical shells built on the core moiety. The core moiety of a dendrimer may be a fluorescent or phosphorescent small molecule emitter. A dendrimer may be a “small molecule,” and it is believed that all dendrimers currently used in the field of OLEDs are small molecules. In general, a small molecule has a well-defined chemical formula with a single molecular weight, whereas a polymer has a chemical formula and a molecular weight that may vary from molecule to molecule.

OLEDs make use of thin organic films that emit light when voltage is applied across the device. OLEDs are becoming an increasingly interesting technology for use in applications such as flat panel displays, illumination, and backlighting. Several OLED materials and configurations are described in U.S. Pat. Nos. 5,844,363, 6,303,238, and 5,707,745, which are incorporated herein by reference in their entirety.

OLED devices are generally (but not always) intended to emit light through at least one of the electrodes, and one or more transparent electrodes may be useful in organic opto-electronic devices. For example, a transparent electrode material, such as indium tin oxide (ITO), may be used as the bottom electrode. A transparent top electrode, such as disclosed in U.S. Pat. Nos. 5,703,436 and 5,707,745, which are incorporated by reference in their entireties, may also be used. For a device intended to emit light only through the bottom electrode, the top electrode does not need to be transparent, and may be comprised of a thick and reflective metal layer having a high electrical conductivity. Similarly, for a device intended to emit light only through the top electrode, the bottom electrode may be opaque and/or reflective. Where an electrode does not need to be transparent, using a thicker layer may provide better conductivity, and using a reflective electrode may increase the amount of light emitted through the other electrode, by reflecting light back towards the transparent electrode. Fully transparent devices may also be fabricated, where both electrodes are transparent. Side emitting OLEDs may also be fabricated, and one or both electrodes may be opaque or reflective in such devices.

As used herein, “top” means furthest away from the substrate, while “bottom” means closest to the substrate. For example, for a device having two electrodes, the bottom electrode is the electrode closest to the substrate, and is generally the first electrode fabricated. The bottom electrode has two surfaces, a bottom surface closest to the substrate, and a top surface further away from the substrate. Where a first layer is described as “disposed over” a second layer, the first layer is disposed further away from substrate. There may be other layers between the first and second layer, unless it is specified that the first layer is “in physical contact with” the second layer. For example, a cathode may be described as “disposed over” an anode, even though there are various organic layers in between.

As used herein, “solution processible” means capable of being dissolved, dispersed, or transported in and/or deposited from a liquid medium, either in solution or suspension form.

As used herein, and as would be generally understood by one skilled in the art, a first “Highest Occupied Molecular Orbital” (HOMO) or “Lowest Unoccupied Molecular Orbital” (LUMO) energy level is “greater than” or “higher than” a second HOMO or LUMO energy level if the first energy level is closer to the vacuum energy level. Since ionization potentials (IP) are measured as a negative energy relative to a vacuum level, a higher HOMO energy level corresponds to an IP having a smaller absolute value (an IP that is less negative). Similarly, a higher LUMO energy level corresponds to an electron affinity (EA) having a smaller absolute value (an EA that is less negative). On a conventional energy level diagram, with the vacuum level at the top, the LUMO energy level of a material is higher than the HOMO energy level of the same material. A “higher” HOMO or LUMO energy level appears closer to the top of such a diagram than a “lower” HOMO or LUMO energy level.

### SUMMARY OF THE INVENTION

An organic light emitting device is provided. The device has an anode, a cathode and an emissive layer disposed between the anode and the cathode. The emissive layer includes a host and a dopant, and the host material is selected from the group consisting of: 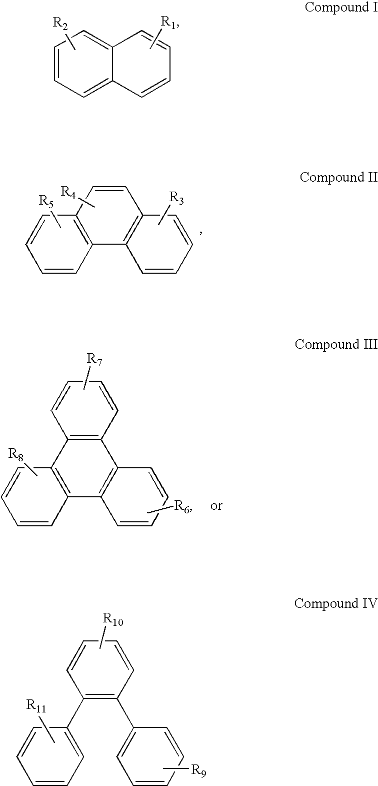
wherein each R represent no substitution, mono-, di-, or tri- substitution, and the substituents are the same or different, and each is selected from the group consisting of alkyl, alkenyl, alkynyl, aryl, thioalkoxy, halo, haloalkyl, cyano, carbonyl, carboxyl, heteroaryl, and substituted aryl, and at least one R for each Compounds I, II, III, or IV includes a carbazole group.

### BRIEF DESCRIPTION OF THE DRAWINGS

FIG. 1 shows an organic light emitting device having separate electron transport, hole transport, and emissive layers, as well as other layers.

FIG. 2 shows an inverted organic light emitting device that does not have a separate electron transport layer.

FIG. 3 shows plots of the current density vs. voltage of devices CuPc(100 Å)/α-NPD(300 Å)/2,7-DCP:Ir(5-Phppy)₃(300 Å, 6%)/BAlq(100 Å)/Alq₃(400 Å) and CuPc(100 Å)/α-NPD(300 Å)/2,7-DCP:Ir(5-Phppy)₃(300 Å, 6%)/HPT(50 Å)/Alq₃(450 Å), and comparative device CuPc(100 Å)/α-NPD(300 Å)/CBP:Ir(5-Phppy)₃(300 Å, 6%)/HPT(50 Å)/Alq₃(450 Å).

FIG. 4 shows plots of external quantum efficiency vs. current density of devices CuPc(100 Å)/α-NPD(300 Å)/2,7-DCP:Ir(5-Phppy)₃(300 Å, 6%)/BAlq(100 Å)/Alq₃(400 Å) and CuPc(100 Å)/α-NPD(300 Å)/2,7-DCP:Ir(5-Phppy)₃(300 Å, 6%)/HPT(50 Å)/Alq₃(450 Å), and comparative device CuPc(100 Å)/α-NPD(300 Å)/CBP:Ir(5-Phppy)₃(300 Å, 6%)/HPT(50 Å)/Alq₃(450 Å).

FIG. 5 shows plots of current density vs. voltage of device CuPc(100 Å)/α-NPD(300 Å)/2,7-DCP:Ir(3′-Meppy)₃(300 Å, 8%)/HPT(50 Å)/Alq₃(450 Å) and comparative device CuPc(100 Å)/α-NPD(300 Å)/CBP:Ir(3′-Meppy)₃(300 Å, 8%)/HPT(50 Å)/Alq₃(450 Å).

FIG. 6 shows plots of external quantum efficiency vs. current density of device CuPc(100 Å)/α-NPD(300 Å)/2,7-DCP:Ir(3′-Meppy)₃(300 Å, 8%)/HPT(50 Å)/Alq₃(450 Å) and comparative device CuPc(100 Å)/α-NPD(300 Å)/CBP:Ir(3′-Meppy)₃(300 Å, 8%)/HPT(50 Å)/Alq₃(450 Å).

FIG. 7 shows plots of current density vs. voltage of devices CuPc(100 Å)/α-NPD(400 Å)/2,7-DCP:Ir(1-piq)₃(300 Å, 12%)/BAlq(100 Å)/Alq₃(500 Å); CuPc(100 Å)/α-NPD(400 Å)/2,7-DCP:Ir(1-piq)₃(300 Å, 12%)/HPT(50 Å)/Alq₃(500 Å); CuPc(100 Å)/α-NPD(400 Å)/2,7-DCP:Ir(1-piq)₃(300 Å, 12%)/Alq₃(500 Å); CuPc(100 Å)/α-NPD(400 Å)/2,7-DCP:Ir(1-piq)₃(300 Å, 6%)/HPT(50 Å)/Alq₃(500 Å); and CuPc(100 Å)/α-NPD(400 Å)/2,7-DCP:Ir(1-piq)₃(300 Å, 8%)/HPT(50 Å)/Alq₃(500 Å).

FIG. 8 show plots of external quantum efficiency vs. current density of devices CuPc(100 Å)/α-NPD(400 Å)/2,7-DCP:Ir(1-piq)₃(300 Å, 12%)/BAlq(100 Å)/Alq₃(500 Å); CuPc(100 Å)/α-NPD(400 Å)/2,7-DCP:Ir(1-piq)₃(300 Å, 12%)/HPT(50 Å)/Alq₃(500 Å); CuPc(100 Å)/α-NPD(400 Å)/2,7-DCP:Ir(1-piq)₃(300 Å, 12%)/Alq₃(500 Å); CuPc(100 Å)/α-NPD(400 Å)/2,7-DCP:Ir(1-piq)₃(300 Å, 6%)/HPT(50 Å)/Alq₃(500 Å); and CuPc(100 Å)/α-NPD(400 Å)/2,7-DCP:Ir(1-piq)₃(300 Å, 8%)/HPT(50 Å)/Alq₃(500 Å).

FIG. 9 shows plots of operation lifetime of device CuPc(100 Å)/α-NPD(300 Å)/2,7-DCP:Ir(5-Phppy)₃(300 Å, 6%)/HPT(50 Å)/Alq₃(450 Å) and comparative device CuPc(100 Å)/α-NPD(300 Å)/CBP:Ir(5-Phppy)₃(300 Å, 6%)/HPT(50 Å)/Alq₃(450 Å).

FIG. 10 shows plots of operation lifetime of device CuPc(100 Å)/α-NPD(300 Å)/2,7-DCP:Ir(3′-Meppy)₃(300 Å, 8%)/HPT(50 Å)/Alq₃(450 Å) and comparative device CuPc(100 Å)/α-NPD(300 Å)/CBP:Ir(3′-Meppy)₃(300 Å, 8%)/HPT(50 Å)/Alq₃(450 Å).

FIG. 11 shows plots of the current density vs. voltage of devices CuPc(100 Å)/α-NPD(300 Å)/3,3′-DC-o-TerP:Ir(5-Phppy)₃(300 Å, 6%)/HPT(50 Å)/Alq₃(450 Å) and CuPc(100 Å)/α-NPD(300 Å)/3,3′-DC-o-TerP:Ir(5-Phppy)₃(300 Å, 6%)/BAlq(100 Å)/Alq₃(450 Å), and CuPc(100 Å)/α-NPD(300 Å)/3,3′-DC-o-TerP:Ir(5-Phppy)₃(300 Å, 6%)/Alq₃(450 Å).

FIG. 12 shows plots of external quantum efficiency vs. current density of devices CuPc(100 Å)/α-NPD(300 Å)/3,3′-DC-o-TerP:Ir(5-Phppy)₃(300 Å, 6%)/HPT(50 Å)/Alq₃(450 Å) and CuPc(100 Å)/α-NPD(300 Å)/3,3′-DC-o-TerP:Ir(5-Phppy)₃(300 Å, 6%)/BAlq(100 A)/Alq₃(450 Å), and CuPc(100 Å)/α-NPD(300 Å)/3,3′-DC-o-TerP:Ir(5-Phppy)₃(300 Å, 6%)/Alq₃(450 Å).

FIG. 13 shows plots of operation lifetime of devices CuPc(100 Å)/α-NPD(300 Å)/3,3′-DC-o-TerP:Ir(5-Phppy)₃(300 Å, 6%)/HPT(50 Å)/Alq₃(450 Å) and CuPc(100 Å)/α-NPD(300 Å)/3,3′-DC-o-TerP:Ir(5-Phppy)₃(300 Å, 6%)/BAlq(100 Å)/Alq₃(450 Å), and CuPc(100 Å)/α-NPD(300 Å)/3,3′-DC-o-TerP:Ir(5-Phppy)₃(300 Å, 6%)/Alq₃(450 Å).

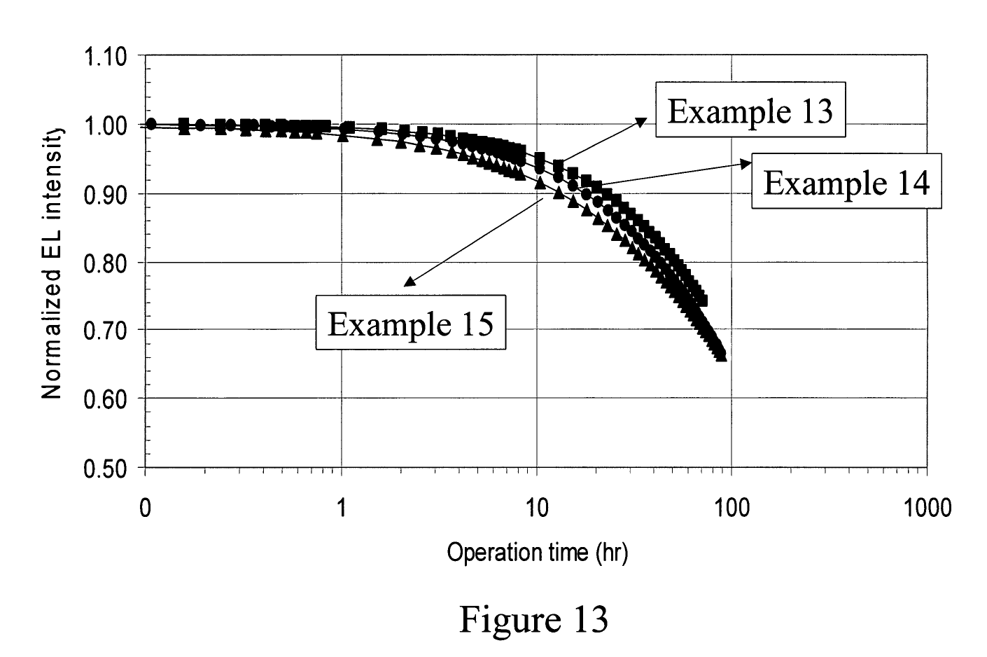

### DETAILED DESCRIPTION

Generally, an OLED comprises at least one organic layer disposed between and electrically connected to an anode and a cathode. When a current is applied, the anode injects holes and the cathode injects electrons into the organic layer(s). The injected holes and electrons each migrate toward the oppositely charged electrode. When an electron and hole localize on the same molecule, an “exciton,” which is a localized electron-hole pair having an excited energy state, is formed. Light is emitted when the exciton relaxes via a photoemissive mechanism. In some cases, the exciton may be localized on an excimer or an exciplex. Non-radiative mechanisms, such as thermal relaxation, may also occur, but are generally considered undesirable.

The initial OLEDs used emissive molecules that emitted light from their singlet states (“fluorescence”) as disclosed, for example, in U.S. Pat. No. 4,769,292, which is incorporated by reference in its entirety. Fluorescent emission generally occurs in a time frame of less than 10 nanoseconds.

More recently, OLEDs having emissive materials that emit light from triplet states (“phosphorescence”) have been demonstrated. Baldo et al., “Highly Efficient Phosphorescent Emission from Organic Electroluminescent Devices,” Nature, vol. 395, 151-154, 1998; (“Baldo-I”) and Baldo et al., “Very high-efficiency green organic light-emitting devices based on electrophosphorescence,” Appl. Phys. Lett., vol. 75, No. 3, 4-6 (1999) (“Baldo-II”), which are incorporated by reference in their entireties. Phosphorescence may be referred to as a “forbidden” transition because the transition requires a change in spin states, and quantum mechanics indicates that such a transition is not favored. As a result, phosphorescence generally occurs in a time frame exceeding at least 10 nanoseconds, and typically greater than 100 nanoseconds. If the natural radiative lifetime of phosphorescence is too long, triplets may decay by a non-radiative mechanism, such that no light is emitted. Organic phosphorescence is also often observed in molecules containing heteroatoms with unshared pairs of electrons at very low temperatures. 2,2′-bipyridine is such a molecule. Non-radiative decay mechanisms are typically temperature dependent, such that an organic material that exhibits phosphorescence at liquid nitrogen temperatures typically does not exhibit phosphorescence at room temperature. But, as demonstrated by Baldo, this problem may be addressed by selecting phosphorescent compounds that do phosphoresce at room temperature. Representative emissive layers include doped or un-doped phosphorescent organo-metallic materials such as disclosed in U.S. Pat. Nos. 6,303,238 and 6,310,360; U.S. Patent Application Publication Nos. 2002-0034656; 2002-0182441; 2003-0072964; and WO-02/074015.

Generally, the excitons in an OLED are believed to be created in a ratio of about 3:1, i.e., approximately 75% triplets and 25% singlets. See, Adachi et al., “Nearly 100% Internal Phosphorescent Efficiency In An Organic Light Emitting Device,” J. Appl. Phys., 90, 5048 (2001), which is incorporated by reference in its entirety. In many cases, singlet excitons may readily transfer their energy to triplet excited states via “intersystem crossing,” whereas triplet excitons may not readily transfer their energy to singlet excited states. As a result, 100% internal quantum efficiency is theoretically possible with phosphorescent OLEDs. In a fluorescent device, the energy of triplet excitons is generally lost to radiationless decay processes that heat-up the device, resulting in much lower internal quantum efficiencies. OLEDs utilizing phosphorescent materials that emit from triplet excited states are disclosed, for example, in U.S. Pat. No. 6,303,238, which is incorporated by reference in its entirety.

Phosphorescence may be preceded by a transition from a triplet excited state to an intermediate non-triplet state from which the emissive decay occurs. For example, organic molecules coordinated to lanthanide elements often phosphoresce from excited states localized on the lanthanide metal. However, such materials do not phosphoresce directly from a triplet excited state but instead emit from an atomic excited state centered on the lanthanide metal ion. The europium diketonate complexes illustrate one group of these types of species.

Phosphorescence from triplets can be enhanced over fluorescence by confining, preferably through bonding, the organic molecule in close proximity to an atom of high atomic number. This phenomenon, called the heavy atom effect, is created by a mechanism known as spin-orbit coupling. Such a phosphorescent transition may be observed from an excited metal-to-ligand charge transfer (MLCT) state of an organometallic molecule such as tris(2-phenylpyridine)iridium(III).

As used herein, the term “triplet energy” refers to an energy corresponding to the highest energy feature discernable in the phosphorescence spectrum of a given material. The highest energy feature is not necessarily the peak having the greatest intensity in the phosphorescence spectrum, and could, for example, be a local maximum of a clear shoulder on the high energy side of such a peak.

The term “organometallic” as used herein is as generally understood by one of ordinary skill in the art and as given, for example, in “Inorganic Chemistry” (2nd Edition) by Gary L. Miessler and Donald A. Tarr, Prentice Hall (1998). Thus, the term organometallic refers to compounds which have an organic group bonded to a metal through a carbon-metal bond. This class does not include per se coordination compounds, which are substances having only donor bonds from heteroatoms, such as metal complexes of amines, halides, pseudohalides (CN, etc.), and the like. In practice, organometallic compounds generally comprise, in addition to one or more carbon-metal bonds to an organic species, one or more donor bonds from a heteroatom. The carbon-metal bond to an organic species refers to a direct bond between a metal and a carbon atom of an organic group, such as phenyl, alkyl, alkenyl, etc., but does not refer to a metal bond to an “inorganic carbon,” such as the carbon of CN or CO.

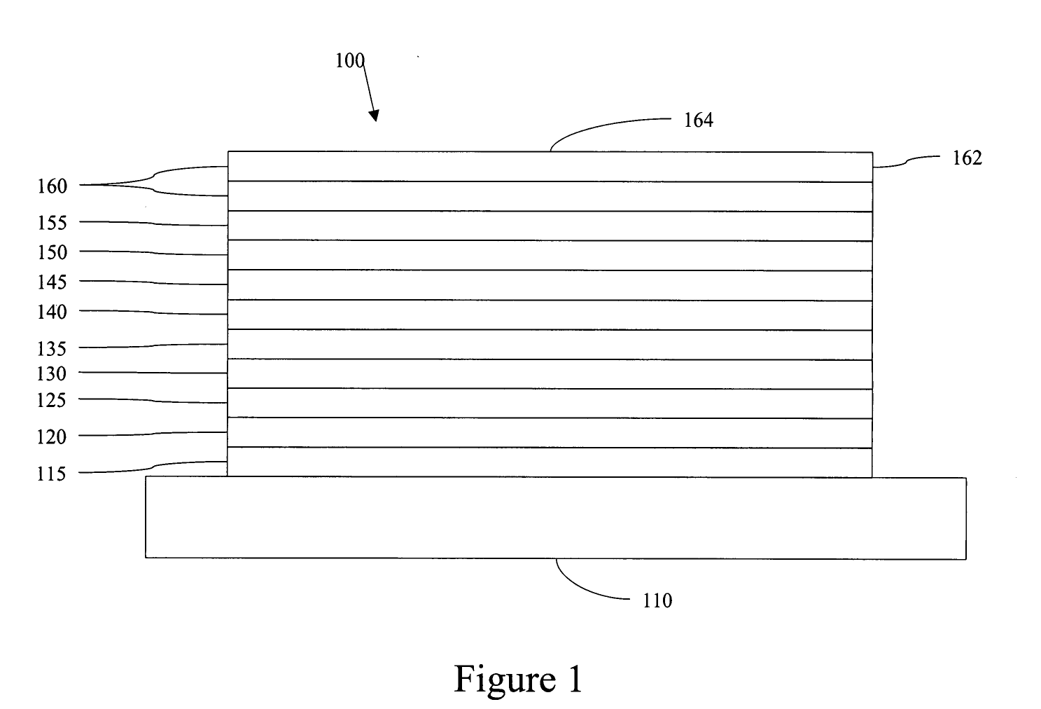

FIG. 1 shows an organic light emitting device 100. The figures are not necessarily drawn to scale. Device 100 may include a substrate 110, an anode 115, a hole injection layer 120, a hole transport layer 125, an electron blocking layer 130, an emissive layer 135, a hole blocking layer 140, an electron transport layer 145, an electron injection layer 150, a protective layer 155, and a cathode 160. Cathode 160 is a compound cathode having a first conductive layer 162 and a second conductive layer 164. Device 100 may be fabricated by depositing the layers described, in order.

Substrate 110 may be any suitable substrate that provides desired structural properties. Substrate 110 may be flexible or rigid. Substrate 110 may be transparent, translucent or opaque. Plastic and glass are examples of preferred rigid substrate materials. Plastic and metal foils are examples of preferred flexible substrate materials. Substrate 110 may be a semiconductor material in order to facilitate the fabrication of circuitry. For example, substrate 110 may be a silicon wafer upon which circuits are fabricated, capable of controlling OLEDs subsequently deposited on the substrate. Other substrates may be used. The material and thickness of substrate 110 may be chosen to obtain desired structural and optical properties.

Anode 115 may be any suitable anode that is sufficiently conductive to transport holes to the organic layers. The material of anode 115 preferably has a work function higher than about 4 eV (a “high work function material”). Preferred anode materials include conductive metal oxides, such as indium tin oxide (ITO) and indium zinc oxide (IZO), aluminum zinc oxide (AlZnO), and metals. Anode 115 (and substrate 110) may be sufficiently transparent to create a bottom-emitting device. A preferred transparent substrate and anode combination is commercially available ITO (anode) deposited on glass or plastic (substrate). A flexible and transparent substrate-anode combination is disclosed in U.S. Pat. Nos. 5,844,363 and 6,602,540 B2, which are incorporated by reference in their entireties. Anode 115 may be opaque and/or reflective. A reflective anode 115 may be preferred for some top-emitting devices, to increase the amount of light emitted from the top of the device. The material and thickness of anode 115 may be chosen to obtain desired conductive and optical properties. Where anode 115 is transparent, there may be a range of thickness for a particular material that is thick enough to provide the desired conductivity, yet thin enough to provide the desired degree of transparency. Other anode materials and structures may be used.

Hole transport layer 125 may include a material capable of transporting holes. Hole transport layer 130 may be intrinsic (undoped), or doped. Doping may be used to enhance conductivity. α-NPD and TPD are examples of intrinsic hole transport layers. An example of a p-doped hole transport layer is m-MTDATA doped with F₄-TCNQ at a molar ratio of 50:1, as disclosed in U.S. Patent Application Publication No. 2003-0230980 to Forrest et al., which is incorporated by reference in its entirety. Other hole transport layers may be used.

Emissive layer 135 may include an organic material capable of emitting light when a current is passed between anode 115 and cathode 160. Preferably, emissive layer 135 contains a phosphorescent emissive material, although fluorescent emissive materials may also be used. Phosphorescent materials are preferred because of the higher luminescent efficiencies associated with such materials. Emissive layer 135 may also comprise a host material capable of transporting electrons and/or holes, doped with an emissive material that may trap electrons, holes, and/or excitons, such that excitons relax from the emissive material via a photoemissive mechanism. Emissive layer 135 may comprise a single material that combines transport and emissive properties. Whether the emissive material is a dopant or a major constituent, emissive layer 135 may comprise other materials, such as dopants that tune the emission of the emissive material. Emissive layer 135 may include a plurality of emissive materials capable of, in combination, emitting a desired spectrum of light. Examples of phosphorescent emissive materials include Ir(ppy)₃. Examples of fluorescent emissive materials include DCM and DMQA. Examples of host materials include Alq₃, CBP and mCP. Examples of emissive and host materials are disclosed in U.S. Pat. No. 6,303,238 to Thompson et al., which is incorporated by reference in its entirety. Emissive material may be included in emissive layer 135 in a number of ways. For example, an emissive small molecule may be incorporated into a polymer. This may be accomplished by several ways: by doping the small molecule into the polymer either as a separate and distinct molecular species; or by incorporating the small molecule into the backbone of the polymer, so as to form a co-polymer; or by bonding the small molecule as a pendant group on the polymer. Other emissive layer materials and structures may be used. For example, a small molecule emissive material may be present as the core of a dendrimer.

Many useful emissive materials include one or more ligands bound to a metal center. A ligand may be referred to as “photoactive” if it contributes directly to the luminescent properties of an organometallic emissive material. A “photoactive” ligand may provide, in conjunction with a metal, the energy levels from which and to which an electron moves when a photon is emitted. Other ligands may be referred to as “ancillary.” Ancillary ligands may modify the photoactive properties of the molecule, for example by shifting the energy levels of a photoactive ligand, but ancillary ligands do not directly provide the energy levels directly involved in light emission. A ligand that is photoactive in one molecule may be ancillary in another. These definitions of photoactive and ancillary are intended as non-limiting theories.

Electron transport layer 145 may include a material capable of transporting electrons. Electron transport layer 145 may be intrinsic (undoped), or doped. Doping may be used to enhance conductivity. Alq₃ is an example of an intrinsic electron transport layer. An example of an n-doped electron transport layer is BPhen doped with Li at a molar ratio of 1:1, as disclosed in U.S. Patent Application Publication No. 2003-0230980 to Forrest et al., which is incorporated by reference in its entirety. Other electron transport layers may be used.

The charge carrying component of the electron transport layer may be selected such that electrons can be efficiently injected from the cathode into the LUMO (Lowest Unoccupied Molecular Orbital) energy level of the electron transport layer. The “charge carrying component” is the material responsible for the LUMO energy level that actually transports electrons. This component may be the base material, or it may be a dopant. The LUMO energy level of an organic material may be generally characterized by the electron affinity of that material and the relative electron injection efficiency of a cathode may be generally characterized in terms of the work function of the cathode material. This means that the preferred properties of an electron transport layer and the adjacent cathode may be specified in terms of the electron affinity of the charge carrying component of the ETL and the work function of the cathode material. In particular, so as to achieve high electron injection efficiency, the work function of the cathode material is preferably not greater than the electron affinity of the charge carrying component of the electron transport layer by more than about 0.75 eV, more preferably, by not more than about 0.5 eV. Similar considerations apply to any layer into which electrons are being injected.

Cathode 160 may be any suitable material or combination of materials known to the art, such that cathode 160 is capable of conducting electrons and injecting them into the organic layers of device 100. Cathode 160 may be transparent or opaque, and may be reflective. Metals and metal oxides are examples of suitable cathode materials. Cathode 160 may be a single layer, or may have a compound structure. FIG. 1 shows a compound cathode 160 having a thin metal layer 162 and a thicker conductive metal oxide layer 164. In a compound cathode, preferred materials for the thicker layer 164 include ITO, IZO, and other materials known to the art. U.S. Pat. Nos. 5,703,436, 5,707,745, 6,548,956 B2, and 6,576,134 B2, which are incorporated by reference in their entireties, disclose examples of cathodes including compound cathodes having a thin layer of metal such as Mg:Ag with an overlying transparent, electrically-conductive, sputter-deposited ITO layer. The part of cathode 160 that is in contact with the underlying organic layer, whether it is a single layer cathode 160, the thin metal layer 162 of a compound cathode, or some other part, is preferably made of a material having a work function lower than about 4 eV (a “low work function material”). Other cathode materials and structures may be used.

Blocking layers may be used to reduce the number of charge carriers (electrons or holes) and/or excitons that leave the emissive layer. An electron blocking layer 130 may be disposed between emissive layer 135 and the hole transport layer 125, to block electrons from leaving emissive layer 135 in the direction of hole transport layer 125. Similarly, a hole blocking layer 140 may be disposed between emissive layer 135 and electron transport layer 145, to block holes from leaving emissive layer 135 in the direction of electron transport layer 145. Blocking layers may also be used to block excitons from diffusing out of the emissive layer. The theory and use of blocking layers is described in more detail in U.S. Pat. No. 6,097,147 and U.S. Patent Application Publication No. 2003-0230980 to Forrest et al., which are incorporated by reference in their entireties.

As used herein, and as would be understood by one of skill in the art, the term “blocking layer” means that the layer provides a barrier that significantly inhibits transport of charge carriers and/or excitons through the device, without suggesting that the layer necessarily completely blocks the charge carriers and/or excitons. The presence of such a blocking layer in a device may result in substantially higher efficiencies as compared to a similar device lacking a blocking layer. Also, a blocking layer may be used to confine emission to a desired region of an OLED.

Generally, injection layers are comprised of a material that may improve the injection of charge carriers from one layer, such as an electrode or an organic layer, into an adjacent organic layer. Injection layers may also perform a charge transport function. In device 100, hole injection layer 120 may be any layer that improves the injection of holes from anode 115 into hole transport layer 125. CuPc is an example of a material that may be used as a hole injection layer from an ITO anode 115, and other anodes. In device 100, electron injection layer 150 may be any layer that improves the injection of electrons into electron transport layer 145. LiF/Al is an example of a material that may be used as an electron injection layer into an electron transport layer from an adjacent layer. Other materials or combinations of materials may be used for injection layers. Depending upon the configuration of a particular device, injection layers may be disposed at locations different than those shown in device 100. More examples of injection layers are provided in U.S. patent application Ser. No. 09/931,948 to Lu et al., which is incorporated by reference in its entirety. A hole injection layer may comprise a solution deposited material, such as a spin-coated polymer, e.g., PEDOT:PSS, or it may be a vapor deposited small molecule material, e.g., CuPc or MTDATA.

A hole injection layer (HIL) may planarize or wet the anode surface so as to provide efficient hole injection from the anode into the hole injecting material. A hole injection layer may also have a charge carrying component having HOMO (Highest Occupied Molecular Orbital) energy levels that favorably match up, as defined by their herein-described relative ionization potential (IP) energies, with the adjacent anode layer on one side of the HIL and the hole transporting layer on the opposite side of the HIL. The “charge carrying component” is the material responsible for the HOMO energy level that actually transports holes. This component may be the base material of the HIL, or it may be a dopant. Using a doped HIL allows the dopant to be selected for its electrical properties, and the host to be selected for morphological properties such as wetting, flexibility, toughness, etc. Preferred properties for the HIL material are such that holes can be efficiently injected from the anode into the HIL material. In particular, the charge carrying component of the HIL preferably has an IP not more than about 0.7 eV greater that the IP of the anode material. More preferably, the charge carrying component has an IP not more than about 0.5 eV greater than the anode material. Similar considerations apply to any layer into which holes are being injected. HIL materials are further distinguished from conventional hole transporting materials that are typically used in the hole transporting layer of an OLED in that such HIL materials may have a hole conductivity that is substantially less than the hole conductivity of conventional hole transporting materials. The thickness of the HIL of the present invention may be thick enough to help planarize or wet the surface of the anode layer. For example, an HIL thickness of as little as 10 nm may be acceptable for a very smooth anode surface. However, since anode surfaces tend to be very rough, a thickness for the HIL of up to 50 nm may be desired in some cases.

A protective layer may be used to protect underlying layers during subsequent fabrication processes. For example, the processes used to fabricate metal or metal oxide top electrodes may damage organic layers, and a protective layer may be used to reduce or eliminate such damage. In device 100, protective layer 155 may reduce damage to underlying organic layers during the fabrication of cathode 160. Preferably, a protective layer has a high carrier mobility for the type of carrier that it transports (electrons in device 100), such that it does not significantly increase the operating voltage of device 100. CuPc, BCP, and various metal phthalocyanines are examples of materials that may be used in protective layers. Other materials or combinations of materials may be used. The thickness of protective layer 155 is preferably thick enough that there is little or no damage to underlying layers due to fabrication processes that occur after organic protective layer 160 is deposited, yet not so thick as to significantly increase the operating voltage of device 100. Protective layer 155 maybe doped to increase its conductivity. For example, a CuPc or BCP protective layer 160 may be doped with Li. A more detailed description of protective layers may be found in U.S. patent application Ser. No. 09/931,948 to Lu et al., which is incorporated by reference in its entirety.

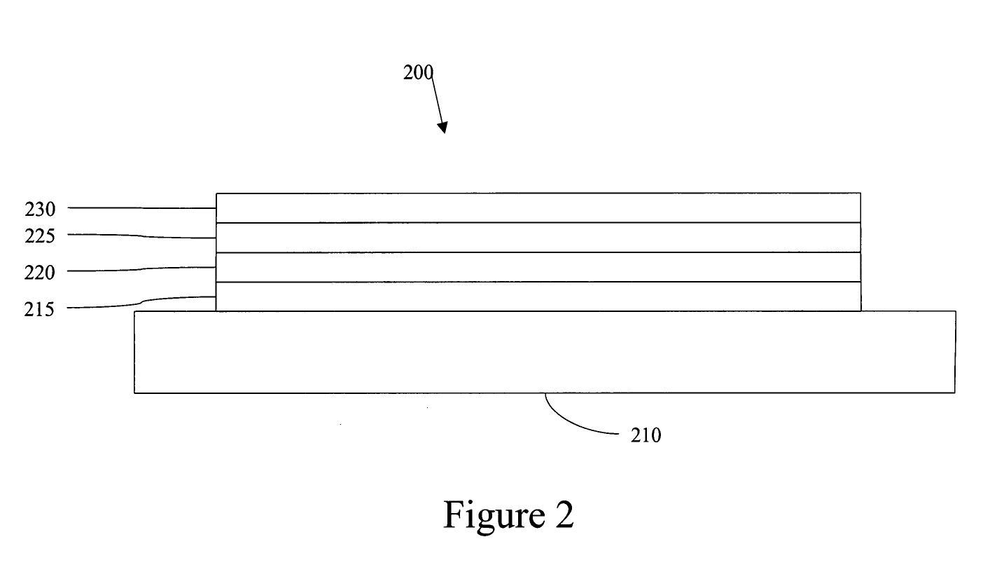

FIG. 2 shows an inverted OLED 200. The device includes a substrate 210, an cathode 215, an emissive layer 220, a hole transport layer 225, and an anode 230. Device 200 may be fabricated by depositing the layers described, in order. Because the most common OLED configuration has a cathode disposed over the anode, and device 200 has cathode 215 disposed under anode 230, device 200 may be referred to as an “inverted” OLED. Materials similar to those described with respect to device 100 may be used in the corresponding layers of device 200. FIG. 2 provides one example of how some layers may be omitted from the structure of device 100.

The simple layered structure illustrated in FIGS. 1 and 2 is provided by way of non-limiting example, and it is understood that embodiments of the invention may be used in connection with a wide variety of other structures. The specific materials and structures described are exemplary in nature, and other materials and structures may be used. Functional OLEDs may be achieved by combining the various layers described in different ways, or layers may be omitted entirely, based on design, performance, and cost factors. Other layers not specifically described may also be included. Materials other than those specifically described may be used. Although many of the examples provided herein describe various layers as comprising a single material, it is understood that combinations of materials, such as a mixture of host and dopant, or more generally a mixture, may be used. Also, the layers may have various sublayers. The names given to the various layers herein are not intended to be strictly limiting. For example, in device 200, hole transport layer 225 transports holes and injects holes into emissive layer 220, and may be described as a hole transport layer or a hole injection layer. In one embodiment, an OLED may be described as having an “organic layer” disposed between a cathode and an anode. This organic layer may comprise a single layer, or may further comprise multiple layers of different organic materials as described, for example, with respect to FIGS. 1 and 2.

Structures and materials not specifically described may also be used, such as OLEDs comprised of polymeric materials (PLEDs) such as disclosed in U.S. Pat. No. 5,247,190, Friend et al., which is incorporated by reference in its entirety. By way of further example, OLEDs having a single organic layer may be used. OLEDs may be stacked, for example as described in U.S. Pat. No. 5,707,745 to Forrest et al, which is incorporated by reference in its entirety. The OLED structure may deviate from the simple layered structure illustrated in FIGS. 1 and 2. For example, the substrate may include an angled reflective surface to improve out-coupling, such as a mesa structure as described in U.S. Pat. No. 6,091,195 to Forrest et al., and/or a pit structure as described in U.S. Pat. No. 5,834,893 to Bulovic et al., which are incorporated by reference in their entireties.

Unless otherwise specified, any of the layers of the various embodiments may be deposited by any suitable method. For the organic layers, preferred methods include thermal evaporation, ink-jet, such as described in U.S. Pat. Nos. 6,013,982 and 6,087,196, which are incorporated by reference in their entireties, organic vapor phase deposition (OVPD), such as described in U.S. Pat. No. 6,337,102 to Forrest et al., which is incorporated by reference in its entirety, and deposition by organic vapor jet printing (OVJP), such as described in U.S. patent application Ser. No. 10/233,470, which is incorporated by reference in its entirety. Other suitable deposition methods include spin coating and other solution based processes. Solution based processes are preferably carried out in nitrogen or an inert atmosphere. For the other layers, preferred methods include thermal evaporation. Preferred patterning methods include deposition through a mask, cold welding such as described in U.S. Pat. Nos. 6,294,398 and 6,468,819, which are incorporated by reference in their entireties, and patterning associated with some of the deposition methods such as ink-jet and OVJD. Other methods may also be used. The materials to be deposited may be modified to make them compatible with a particular deposition method. For example, substituents such as alkyl and aryl groups, branched or unbranched, and preferably containing at least 3 carbons, may be used in small molecules to enhance their ability to undergo solution processing. Substituents having 20 carbons or more may be used, and 3-20 carbons is a preferred range. Materials with asymmetric structures may have better solution processibility than those having symmetric structures, because asymmetric materials may have a lower tendency to recrystallize. Dendrimer substituents may be used to enhance the ability of small molecules to undergo solution processing.

The molecules disclosed herein may be substituted in a number of different ways without departing from the scope of the invention. For example, substituents may be added to a compound having three bidentate ligands, such that after the substituents are added, one or more of the bidentate ligands are linked together to form, for example, a tetradentate or hexadentate ligand. Other such linkages may be formed. It is believed that this type of linking may increase stability relative to a similar compound without linking, due to what is generally understood in the art as a “chelating effect.”

Devices fabricated in accordance with embodiments of the invention may be incorporated into a wide variety of consumer products, including flat panel displays, computer monitors, televisions, billboards, lights for interior or exterior illumination and/or signaling, heads up displays, fully transparent displays, flexible displays, laser printers, telephones, cell phones, personal digital assistants (PDAs), laptop computers, digital cameras, camcorders, viewfinders, micro-displays, vehicles, a large area wall, theater or stadium screen, or a sign. Various control mechanisms may be used to control devices fabricated in accordance with the present invention, including passive matrix and active matrix. Many of the devices are intended for use in a temperature range comfortable to humans, such as 18 degrees C. to 30 degrees C., and more preferably at room temperature (20-25 degrees C.).

The materials and structures described herein may have applications in devices other than OLEDs. For example, other optoelectronic devices such as organic solar cells and organic photodetectors may employ the materials and structures. More generally, organic devices, such as organic transistors, may employ the materials and structures.

The term “halo” or “halogen” as used herein includes fluorine, chlorine, bromine and iodine.

The term “alkyl” as used herein contemplates both straight and branched chain alkyl radicals. Preferred alkyl groups are those containing from one to fifteen carbon atoms and includes methyl, ethyl, propyl, isopropyl, butyl, isobutyl, tert-butyl, and the like. Additionally, the alkyl group may be optionally substituted with one or more substituents selected from halo, CN, CO₂R, C(O)R, NR₂, cyclic-amino, NO₂, and OR.

The term “cycloalkyl” as used herein contemplates cyclic alkyl radicals. Preferred cycloalkyl groups are those containing 3 to 7 carbon atoms and includes cyclopropyl, cyclopentyl, cyclohexyl, and the like. Additionally, the cycloalkyl group may be optionally substituted with one or more substituents selected from halo, CN, CO₂R, C(O)R, NR₂, cyclic-amino, NO₂, and OR.

The term “alkenyl” as used herein contemplates both straight and branched chain alkene radicals. Preferred alkenyl groups are those containing two to fifteen carbon atoms. Additionally, the alkenyl group may be optionally substituted with one or more substituents selected from halo, CN, CO₂R, C(O)R, NR₂, cyclic-amino, NO₂, and OR.

The term “alkynyl” as used herein contemplates both straight and branched chain alkyne radicals. Preferred alkyl groups are those containing two to fifteen carbon atoms. Additionally, the alkynyl group may be optionally substituted with one or more substituents selected from halo, CN, CO₂R, C(O)R, NR₂, cyclic-amino, NO₂, and OR.

The term “heterocyclic group” as used herein contemplates non-aromatic cyclic radicals. Preferred heterocyclic groups are those containing 3 or 7 ring atoms which includes at least one hetero atom, and includes cyclic amines such as morpholino, piperdino, pyrrolidino, and the like, and cyclic ethers, such as tetrahydrofuran, tetrahydropyran, and the like.

The term “aryl” or “aromatic group” as used herein contemplates single-ring groups and polycyclic ring systems. The polycyclic rings may have two or more rings in which two atoms are common by two adjoining rings (the rings are “fused”) wherein at least one of the rings is aromatic, e.g., the other rings can be cycloalkyls, cycloalkenyls, aryl, heterocycles and/or heteroaryls.

The term “heteroaryl” as used herein contemplates single-ring hetero-aromatic groups that may include from one to four heteroatoms, for example, pyrrole, furan, thiophene, imidazole, oxazole, thiazole, triazole, tetrazole, pyrazole, pyridine, pyrazine and pyrimidine, and the like. The term heteroaryl also includes polycyclic hetero-aromatic systems having two or more rings in which two atoms are common to two adjoining rings (the rings are “fused”) wherein at least one of the rings is a heteroaryl, e.g., the other rings can be cycloalkyls, cycloalkenyls, aryl, heterocycles and/or heteroaryls.

In embodiments of the invention, host materials of the emissive layer include arylcarbazole complexes with a high degree of π-conjugation. Preferably, the host materials are mono, di or tri carbazole substituted o-terphenyl, triphenylene, and napthalene compounds.

Arylcarbazoles, such as CBP and mCP, may be used as host materials due to favorable charge transport properties. These materials are believed to be good hole injectors and may also provide for reasonable mobility for both holes and electrons. In arylcarbazoles, the carbazole moiety, on which the HOMO is mainly localized, is believed to primarily facilitate hole transport. The electron transport property is believed to be dependent on the aryl core, on which the LUMO is mainly localized, and to which the carbazole is attached.

Host materials of embodiments of the invention include aryl cores having a higher degree of π-conjugation than CBP or mCP while retaining high triplet energy levels. The π-conjugation of arylcarbazoles may be increased by extending the π-conjugation by fusing aryl rings or extending the double bonds by e.g. ortho or para substitutions. It is believed that the oxidized (cation radical) and reduced (anion radical) states of organic materials with high degree of π-conjugation have higher stability than the less conjugated ones. This may be because in the charged state the hole or electron can delocalize more extensively. Where the core of arylcarbazoles have a higher degree of π-conjugation, it is believed to stabilize the reduced (anion radical) state, resulting in more stable electron transport. It is therefore expected that device operation lifetime may be enhanced by incorporating host materials with increased π-conjugation, as compared to CBP and mCP, in the emissive layer.

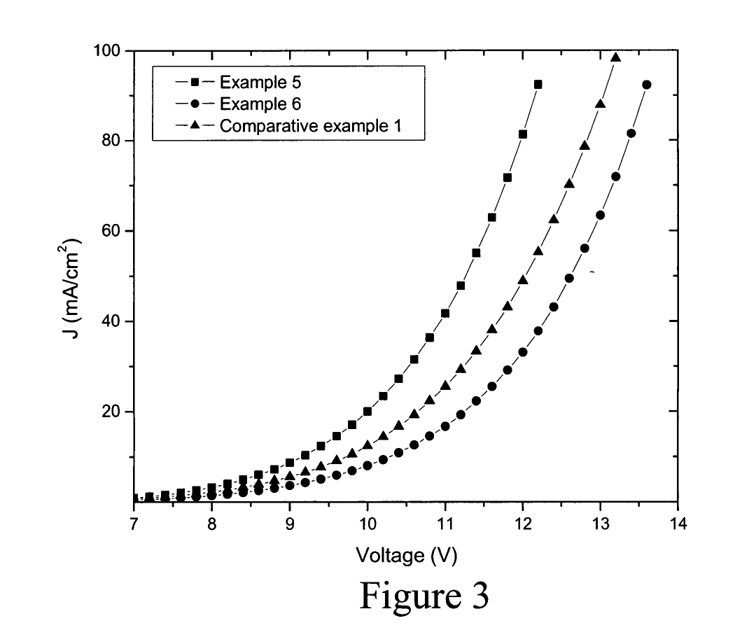

FIGS. 3-8 show that devices with 2,7-DCP as the host material exhibit higher stability than the CBP host devices. It is believed that the higher stability results from the phenanthrene core of 2,7-DCP which is more conjugated than the biphenyl core of CBP.

The degree of π-conjugation also affects the HOMO and LUMO properties of compounds. Generally, increasing the degree of π-conjugation also decreases the ban gap (i.e. the energy difference between the LUMO and HOMO level is smaller), which also corresponds to a lower triplet energy. The HOMO properties of arylcarbazoles are believed to be localized on the carbazole while the LUMO properties are localized in the aryl cores. Increasing the degree of π-conjugation of an aryl compound, for example from a biphenyl to a napthalene, lowers the LUMO level thus decreasing the band gap, which could lead to quenching if the band gap becomes too small. Embodiments of the invention are believed to possess sufficiently high triplet energy levels for use in blue, green, red, and white OLEDs.

The following host materials are provided: 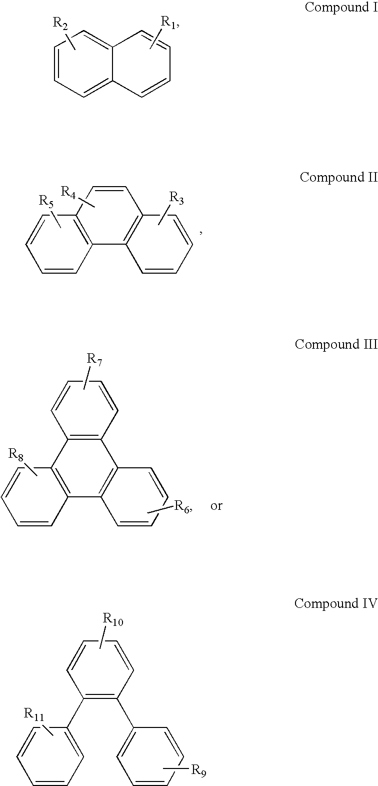
wherein each R represent no substitution, mono-, di-, or tri- substitution, and the substituents are the same or different, and may be alkyl, alkenyl, alkynyl, aryl, thioalkoxy, halo, haloalkyl, cyano, carbonyl, carboxyl, heteroaryl, and substituted aryl, and at least one R for each Compounds I, II, III, or IV includes a carbazole group.

It is believed that the o-terphenyl, triphenylene, napthalene, and phenanthrene cores shown in Compounds I-IV above would be particularly useful as host materials because they have a higher degree of π-conjugation than commonly used host material cores, such as biphenyl and benzene, and their triplet energy is sufficiently high for blue-green, green, and red PHOLED applications.

In addition, regioisomers of mono, di or tri carbazole substituted o-terphenyl, triphenylene, naphthalene, and phenanthrene are also provided. Substitutions of three or less carbazole units are preferred, because substitution with four or more carbazole units may result in a compound that are difficult to sublime due to large molecular weight. Substitutions of three or less carbazole units may also be preferred, because substituting with four or more carbazole units may result in a compound with solubility in commonly used organic solvents that is too low for convenient solution processing. Substitution with four or more carbazole units is less preferred, but may be useful in some circumstances.

The R substituents in the above Compounds I-IV, which are generally electron donating substituents, are believed to raise the HOMO levels of the compounds. Carbazoles are preferred substituents due to the favorable charge transport properties. Certain substituents, however, may have the effect of increasing HOMO levels to an extent that the materials are less effective at inducing the phosphorescent dopant trapping holes, thereby decreasing device efficiency. For these reasons, some strong electron donating groups, for example, alkoxy or amino groups, are less desirable host substituents.

In a preferred embodiment, each R represents no substitution, mono-, di-, or tri-substitution and all substituents are carbazole.

In one embodiment, the host material includes a compound with the formula: 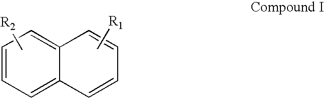
Preferably, R₁ and/or R₂ include a carbazole group. More preferably, R₁ and R₂ each include a carbazole group.

In a preferred embodiment, the host material includes a compound with the formula:

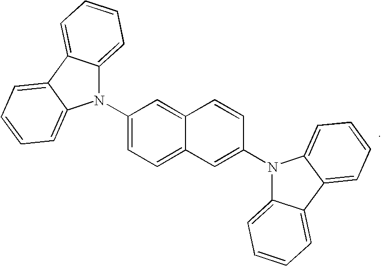

In another embodiment, the host material includes a compound with the formula: 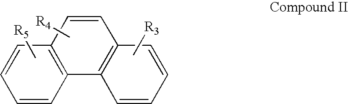
Preferably, at least one of R₃, R₄, and R₅ includes a carbazole group. More preferably, R₃ and R₅ each include a carbazole group.

In a preferred embodiment, the host material includes a compound with the formula:

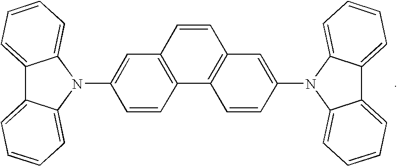

In another embodiment, the host material includes a compound with the formula: 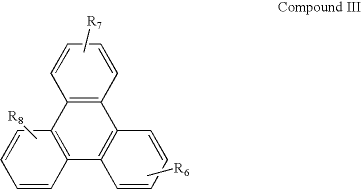
Preferably, at least one of R₆, R₇, and R₈ includes a carbazole group. More preferably, R₆ and R₈ each include a carbazole group.

In a preferred embodiment, the host material includes a compound with the formula:

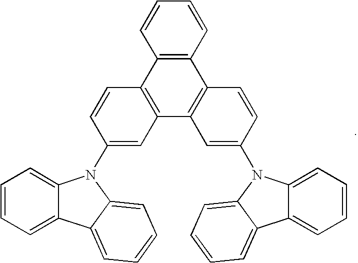

In another preferred embodiment, the host material includes a compound with the formula:

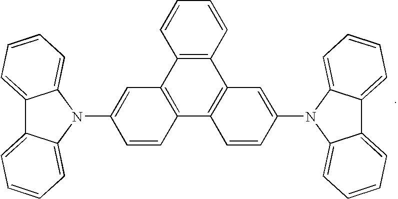

In another embodiment, the host material includes a compound with the formula: 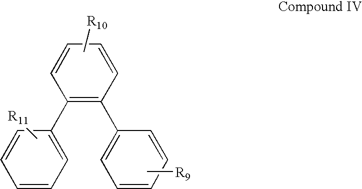
Preferably, at least one of R₉, R₁₀, and R₁₁ includes a carbazole group. More preferably, R₉ and R₁₁ each include a carbazole group.

In a preferred embodiment, the host material includes a compound with the formula:

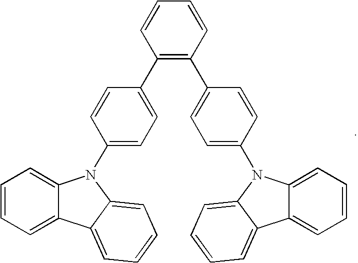

In another preferred embodiment, the host material includes a compound with the formula:

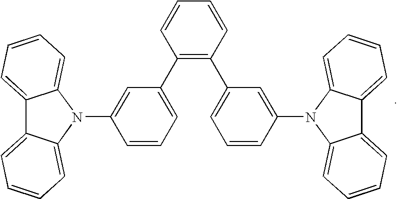

It is understood that the various embodiments described herein are by way of example only, and are not intended to limit the scope of the invention. For example, many of the materials and structures described herein may be substituted with other materials and structures without deviating from the spirit of the invention. It is understood that various theories as to why the invention works are not intended to be limiting. For example, theories relating to charge transfer are not intended to be limiting.

Material Definitions:

As used herein, abbreviations refer to materials as follows:
CBP: 4,4′-N,N-dicarbazole-biphenyl
m-MTDATA 4,4′,4″-tris(3-methylphenylphenlyamino)triphenylamine
Alq₃: 8-tris-hydroxyquinoline aluminum
Bphen: 4,7-diphenyl- 1,10-phenanthroline
n-BPhen: n-doped BPhen (doped with lithium)
F₄-TCNQ: tetrafluoro-tetracyano-quinodimethane
p-MTDATA: p-doped m-MTDATA (doped with F₄-TCNQ)
Ir(Ppy)₃: tris(2-phenylpyridine)-iridium
Ir(ppz)₃: tris(1-phenylpyrazoloto,N,C(2′)iridium(III)
BCP: 2,9-dimethyl-4,7-diphenyl-1,10-phenanthroline
TAZ: 3-phenyl-4-(1′-naphthyl)-5-phenyl-1,2,4-triazole
CuPc: copper phthalocyanine.
ITO: indium tin oxide
NPD: N,N′-diphenyl-N-N′-di(1-naphthyl)-benzidine
TPD: N,N′-diphenyl-N-N′-di(3-toly)-benzidine
BAlq: aluminum(III)bis(2-methyl-8-hydroxyquinolinato)4-phenylphenolate
mCP: 1,3-N,N-dicarbazole-benzene
DCM: 4-(dicyanoethylene)-6-(4-dimethylaminostyryl-2-methyl)-4H-pyran
DMQA: N,N′-dimethylquinacridone
PEDOT:PSS: an aqueous dispersion of poly(3,4-ethylenedioxythiophene) with polystyrenesulfonate (PSS)
HPT: 2,3,6,7,10,11-hexaphenyltriphenylene
2,7-DCP 2,7-N,N-dicarbazolephenanthrene
3,3′-DC-o-TerP 3,3′-dicarbazole-o-terphenyl
4,4′-DC-o-TerP 4,4′-dicarbazole-o-terphenyl
2,6′-DCN 2,6-N,N-dicarbazolenaphthalene
Ir(5-Phppy)₃ tris[5-phenyl(2-phenylpyridine)]iridium(III)
Ir(3′-Meppy)3: tris(3-methyl-2-phenylpyridine) iridium(III)
Ir(1-piq)₃ tris(1-phenylisoquinoline)iridium(III)

### EXPERIMENTAL

Specific representative embodiments of the invention will now be described, including how such embodiments may be made. It is understood that the specific methods, materials, conditions, process parameters, apparatus and the like do not necessarily limit the scope of the invention.

### SYNTHESIS OF ARYLCARBAZOLE COMPLEXES

### EXAMPLE 1

### Synthesis of 2,7-N,N-dicarbazolephenanthrene

Step 1: Synthesis of 2,7-Dibromodihydrophenanthrene

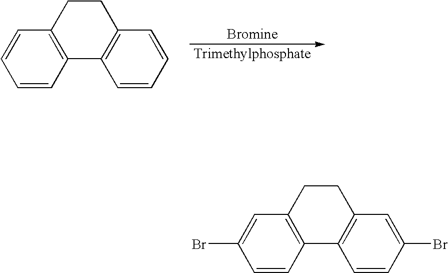

Dihydrophenanthrene (20 g, 0.105 mol) and 120 mL of trimethyl phosphate were dissolved together in a 500 mL round bottom flask. Bromine (38.0 g, 0.235 mol) was dissolved in 80 mL of trimethyl phosphate and the solution was slowly added to the reaction mixture and stirred at room temperature overnight. The reaction mixture was placed in the refrigerator (˜−10° C.) for two days. A precipitate formed and the crude product was vacuumed filtered and then washed with cold ethanol. The crude solid was recrystallized in chloroform to give 2,7-dibromodihydrophenanthrene (25 grams) as a white solid.
Step 2: Synthesis of 2,7-Dibromophenanthrene

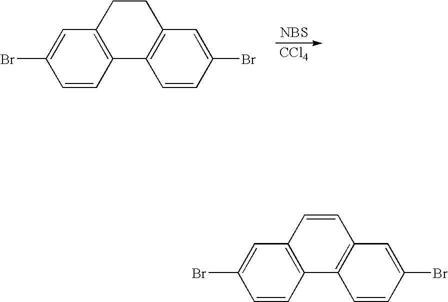

2,7-Dibromodihydrophenanthrene (4.0 g, 0.071 mol), NBS (12.7 g, 0.072 mol) were mixed with 250 mL CCl₄. The mixture was refluxed for 3 hours and cooled down to filter. The solid was washed with hexane/acetone mixture to give 2,7-dibromophenanthrene (23.0 g) as a white solid.
Step 3: Synthesis of 2,7-N,N-dicarbazolephenanthrene

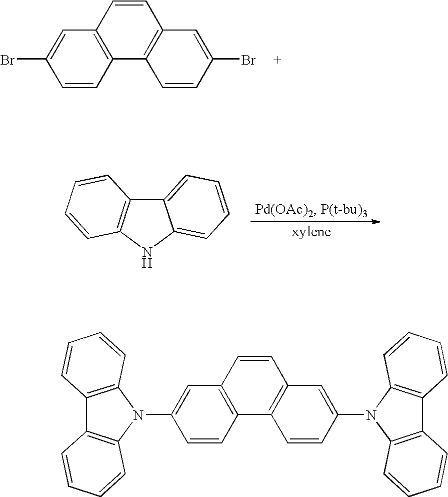

2,7-Dibromophenanthrene (8.0 g, 0.0238 mol), carbazole (8.38 g, 0.050 mol) palladium acetate (0.165 g, 0.00073 mol), tri-t-butylphosphine (0.5 g, 0.0024 mol), sodium ter-butoxide (9.5 g, 0.099 mol) and 120 mL o-xylene were added in a 250 mL round bottom flask. The mixture was refluxed under nitrogen atmosphere overnight and then cooled down to filter crude product. The crude solid was purified by using silica gel column chromatography to give 2,7-N,N-dicarbozalephenanthrene (8.5 grams) as a white solid.

### EXAMPLE 2

### Synthesis of 2,6-N,N-dicarbazolenaphthalene

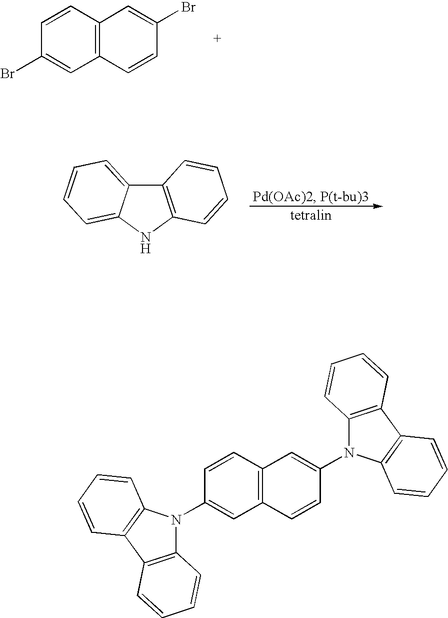

2,6-Dibromonaphthalene(5.0 g, 17.5 mmoles) and carbazole (6.4 g 38.5 mmoles) were dissolved together in tetralin to which palladium acetate (II) (0.12 g, 0.5 mmole) tributylphosphine (0.35 g, 1.7 mmols) and sodium tert-butoxide (6.9 g, 72 mmoles) were added. The reaction mixture was heated to reflux for 48 hours and allowed to cool. The crude material was dissolved into methylene chloride and washed with water, followed by brine. The organic layer was separated and dried over magnesium sulfate, concentrated and purified on a silica gel column using ethyl acetate and hexanes as the eluants. The purified product was isolated to give 2,6-dicarbazolenaphthalene (3.0 grams).

### EXAMPLE 3

### Synthesis of 3,3′-dicarbazole-o-terphenyl

Step 1: Synthesis of 3,3′-N,N-dibromo-o-terphenyl

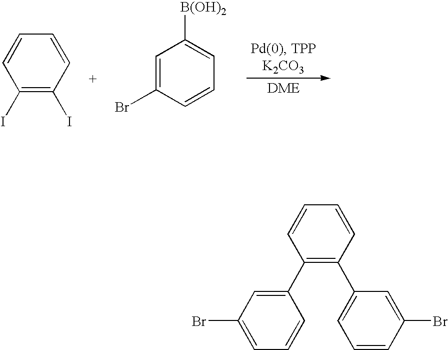

A solution of 1,2-diiodobenzene (5.0 g, 15.2 mmol), 3-bromophenylboronic acid (6.39 g, 31.8 mmol), palladium (II) acetate (0.172 g, 0.764 mmol), triphenylphosphine (0.796 g, 3.0 mmol), and potassium carbonate (5.66 g, 41.0 mmol) in 31 mL of dimethoxyethane and 20 mL of water was heated at reflux under a nitrogen atmosphere for 20 hours, after which an additional 4.0 g (19.9 mmol) of 3-bromophenylboronic acid was added. The solution was maintained at reflux for 20 more hours, cooled, and diluted with ethyl acetate. The aqueous phase was discarded, and after drying over magnesium sulfate, the organic was evaporated off. The crude product was collected by vacuum distillation at 250° C., giving 5.0 g of a crude yellow solid that was recrystallized from absolute ethanol to yield 3,3′-dibromo-o-terphenyl (2.5 g, 42.4%) as white needles.
Step 2: Synthesis of 3,3′-N,N-dicarbazole-o-terphenyl

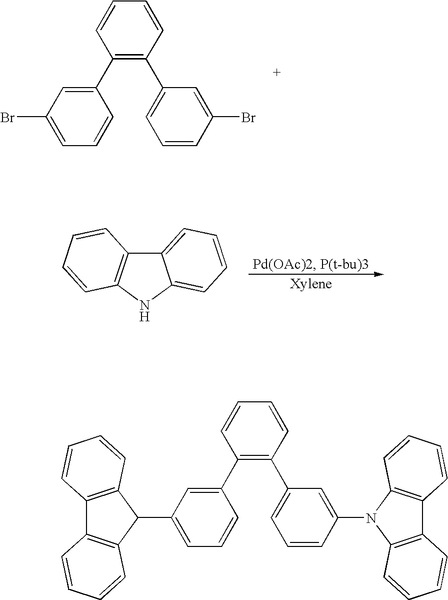

3,3′-dibromo-o-terphenyl (4.9 g, 12.6 mmol), carbazole (4.5 g, 26.9 mmol), palladium (II) acetate (0.087 g, 0.387 mmol), tri(t-butyl)phenylphosphine (0.260 g, 1.29 mmol), and sodium t-butoxide (6.2 g, 64.6 mmol) in 40 mL of xylene was heated under nitrogen at reflux for 40 hours. Additional carbazole (2.0 g, 12 mmol) and catalyst (0.03 eq) were added and the solution was heated. After an additional 60 hours of heating, the solvent (xylene) was evaporated off and replaced with tetralin. The solution was cooled, and the solids were collected by vacuum filtration and washed with methylene chloride. The methylene chloride and tetralin solutions were combined and evaporated down to yield an off-white powder which was combined with methanol and collected by vacuum filtration. The crude product was purified on a silica gel column using 50/50 dichloromethane/hexane as the eluent, resulting in 1.7 g of a white powder that was recrystallized from 40/60 ethyl acetate/hexane to afford 1.1 g (15.5%) of 3,3′-dicarbazole-o-terphenyl as white needles.

### EXAMPLE 4

### Synthesis of 4,4′-N,N-dicarbazole-o-terphenyl

Step 1: Synthesis of 4,4′-dibromo-o-terphenyl

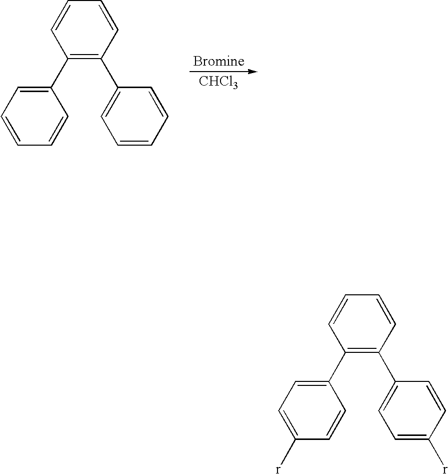

A solution of bromine (89.3 g, 559 mmol) in 90 mL of chloroform was added dropwise over 2.5 hours from an addition funnel to a two liter, three-necked, round bottom flask connected to a sodium bicarbonate trap and charged with a solution of 290 mL of chloroform and 61.3 g (266 mmol) of o-terphenyl. After completion of the addition, the solution was stirred for an additional two hours. Ice and a 4 N aqueous sodium hydroxide solution were then added until the solution became basic. The aqueous phase was discarded, and the organic phase was washed with water, dried over magnesium sulfate and evaporated to dryness. The resulting sticky white powder was stirred with hexane and collected by filtration to afford 53.8 g of a white powder. The crude product (51.3 g) was recrystallized five times from hexane to yield 15.0 g (15%) of pure 4,4′-dibromo-o-terphenyl as fine white crystals.
Step 2: Synthesis of 4,4′-N,N-dicarbazole-o-terphenyl

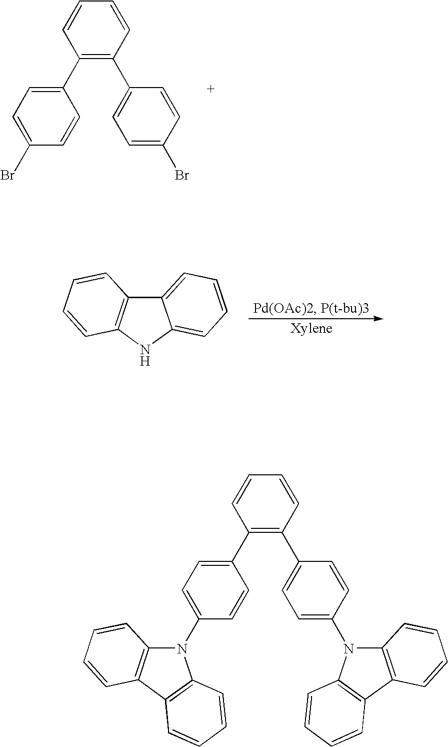

A solution of 4,4′-dibromo-o-terphenyl (15.0 g, 38.7 mmol), carbazole (13.6 g, 81.4 mmol), palladium (II) acetate (0.261 g, 1.2 mmol), tri(t-butyl)phosphine (0.781 g, 3.9 mmol), and sodium t-butoxide (18.6 g, 193 mmol) in 120 mL of xylene was heated at reflux under a nitrogen atmosphere for 20 h. The precipitate was collected by vacuum filtration and extracted with methylene chloride. Addition of methanol to the xylene solution resulted in a white precipitate, which was filtered and added to the methylene chloride extract. 4,4′-dicarbazole-o-terphenyl (12.0 g, 65%) was precipitated out of the methylene chloride solution by the addition of methanol. The product (2.0 g) was further purified using flash chromatography with a 10/90 ethyl acetate/hexane to 100% ethyl acetate solvent gradient, yielding 1.5 g material that was then recrystallized from 160 mL of 25/75 dichloroethane/heptane to give 1.0 g of white needles.

Device fabrication and measurement

All devices were fabricated by high vacuum (<10⁻⁷ Torr) thermal evaporation. The anode electrode was ˜1200 Å of indium tin oxide (ITO). The cathode consisted of 10 Å of LiF followed by 1,000 Å of A1. All devices were encapsulated with a glass lid sealed with an epoxy resin in a nitrogen glove box (<1 ppm of H₂O and O₂) immediately after fabrication, and a moisture getter was incorporated inside the package. The devices consisted of either one electron transporting layer layer (ETL2) or two ETL layers (ETL2 and ETL1). ETL2 refers to the ETL adjacent to the emissive layer (EML) and ETL1 refers to the ETL adjacent to ETL2.

### EXAMPLE 5

The organic stack consisted of sequentially, from the ITO surface, 100 Å thick of copper phthalocyanine (CuPc) as the hole injection layer (HIL), 300 Å of 4,4′-bis[N-(1-naphthyl)-N-phenylamino]biphenyl (α-NPD), as the hole transporting layer (HTL), 300 Å of 2,7-N,N-dicarbazolephenanthrene (2,7-DCP) doped with 6 wt % of Ir(5-Phppy)₃ as the emissive layer (EML), 100 Å of aluminum(III)bis(2-methyl-8-hydroxyquinolinato)4-phenylphenolate (BAlq) as the ETL2, and 400 Å of tris(8-hydroxyquinolinato)aluminum (Alq₃) as the ETL1.

### EXAMPLE 6

The organic stack consisted of sequentially, from the ITO surface, 100 Å thick of copper phthalocyanine (CuPc) as the hole injection layer (HIL), 300 Å of 4,4′-bis[N-(1-naphthyl)-N-phenylamino]biphenyl (α-NPD), as the hole transporting layer (HTL), 300 Å of 2,7-N,N-dicarbazolephenanthrene (2,7-DCP) doped with 6 wt % of Ir(5-Phppy)₃ as the emissive layer (EML), 50 Å of 2,3,6,7,10,11-hexaphenyltriphenylene (HPT) as the ETL2, and 450 Å of tris(8-hydroxyquinolinato)aluminum (Alq₃) as the ETL1.

### Comparative Example 1

The organic stack consisted of sequentially, from the ITO surface, 100 Å thick of copper phthalocyanine (CuPc) as the hole injection layer (HIL), 300 Å of 4,4′-bis[N-(1-naphthyl)-N-phenylamino]biphenyl (α-NPD) as the hole transporting layer (HTL), 300 Å of 4,4′-bis(N-carbazolyl)biphenyl (CBP) doped with 6 wt % of Ir(5-Phppy)₃ as the emissive layer (EML), 50 Å of 2,3,6,7,10,11-hexaphenyltriphenylene (HPT) as the ETL2, and 450 A of tris(8-hydroxyquinolinato)aluminum (Alq₃) as the ETL1.

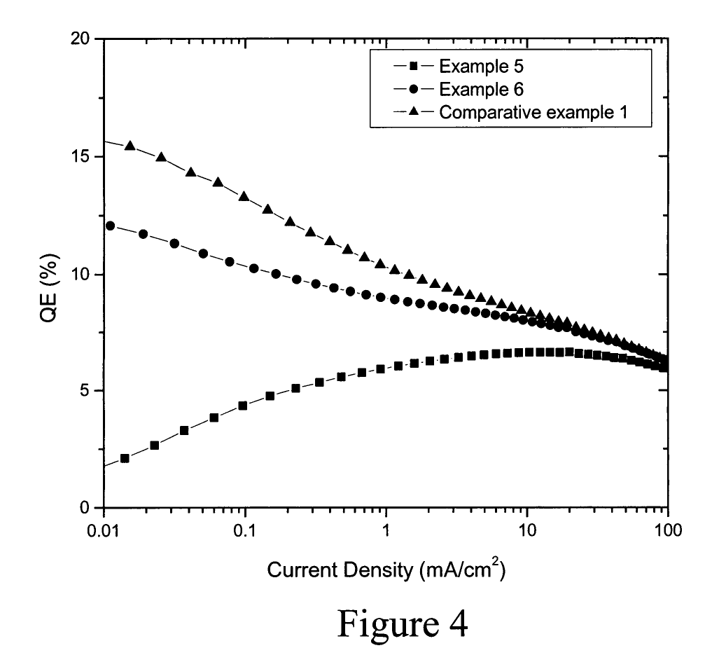

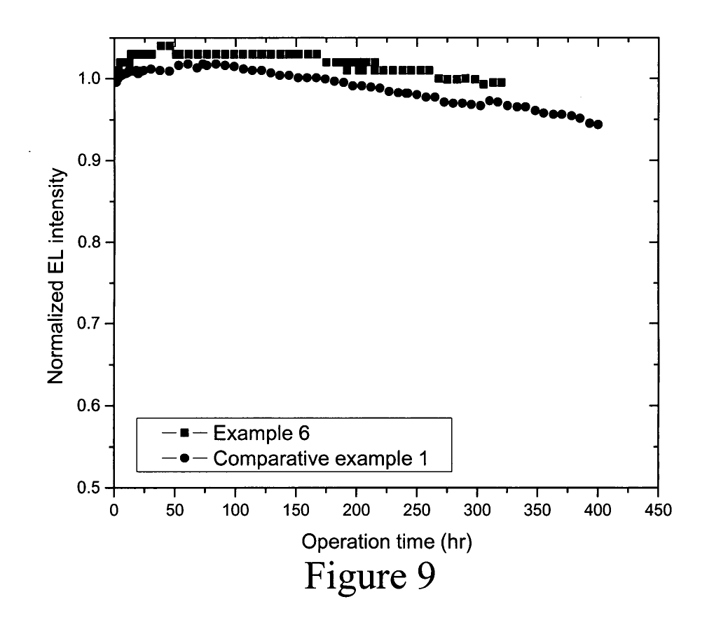

FIG. 3 shows plots of the current density vs. voltage of device Examples 5 and 6, and Comparative Example 1. FIG. 4 shows plots of external quantum efficiency vs. current density of devices Examples 5 and 6, and Comparative Example 1. FIG. 9 shows plots of operation lifetime of device Example 6 and Comparative Example 1.

### EXAMPLE 7

The organic stack consisted of sequentially, from the ITO surface, 100 Å thick of copperphthalocyanine (CuPc) as the hole injection layer (HIL), 300 Å of 4,4′-bis[N-(1-naphthyl)-N-phenylamino]biphenyl (α-NPD), as the hole transporting layer (HTL), 300 Å of 2,7-N,N-dicarbazolephenanthrene (2,7-DCP) doped with 8 wt % of Ir(3′-Meppy)₃ as the emissive layer (EML), 50 Å of 2,3,6,7,10,11-hexaphenyltriphenylene (HPT) as the ETL2, and 450 Å of tris(8-hydroxyquinolinato)aluminum (Alq₃) as the ETL1.

### Comparative Example 2

The organic stack consisted of sequentially, from the ITO surface, 100 Å thick of copper phthalocyanine (CuPc) as the hole injection layer (HIL), 300 Å of 4,4′-bis[N-(1-naphthyl)-N-phenylamino]biphenyl (α-NPD) as the hole transporting layer (HTL), 300 Å of 4,4′-bis(N-carbazolyl)biphenyl (CBP) doped with 8 wt % of Ir(3′-Meppy)₃ as the emissive layer (EML), 50 Å of 2,3,6,7,10,11-hexaphenyltriphenylene (HPT) as the ETL2, and 450 Å of tris(8-hydroxyquinolinato)aluminum (Alq₃) as the ETL1.

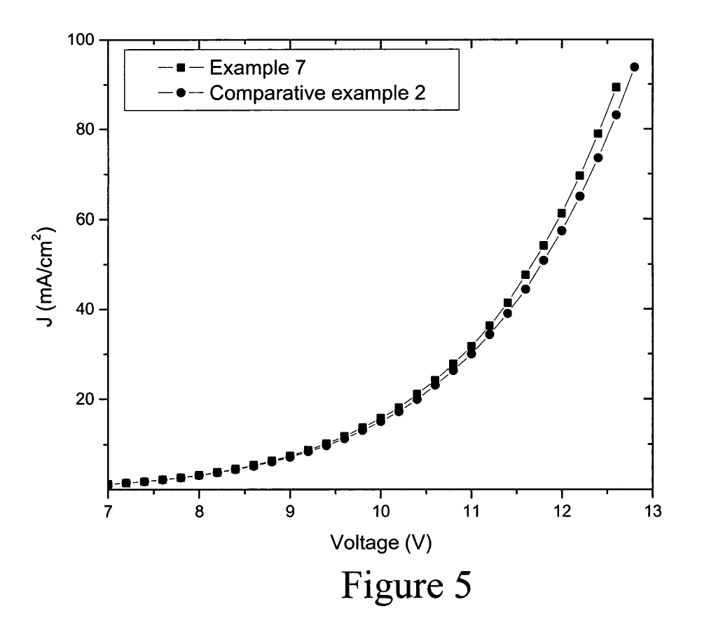

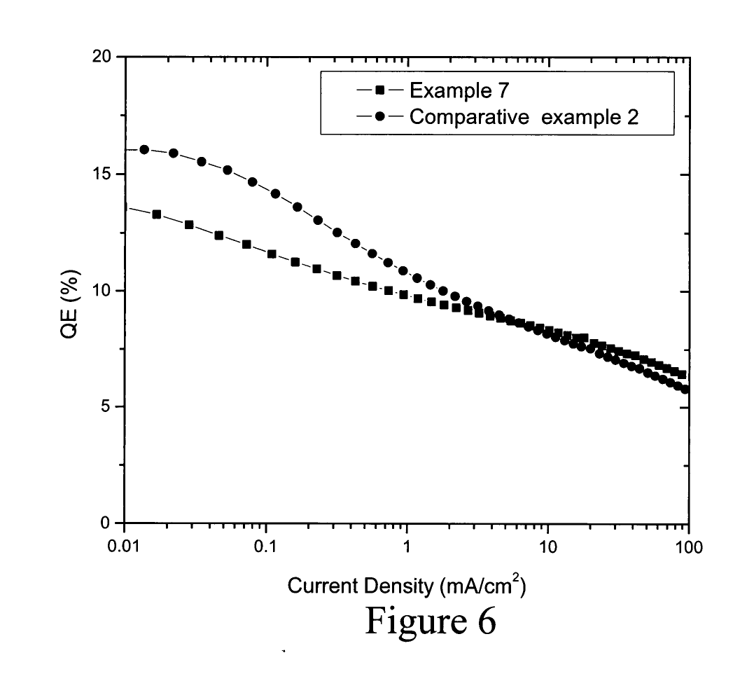

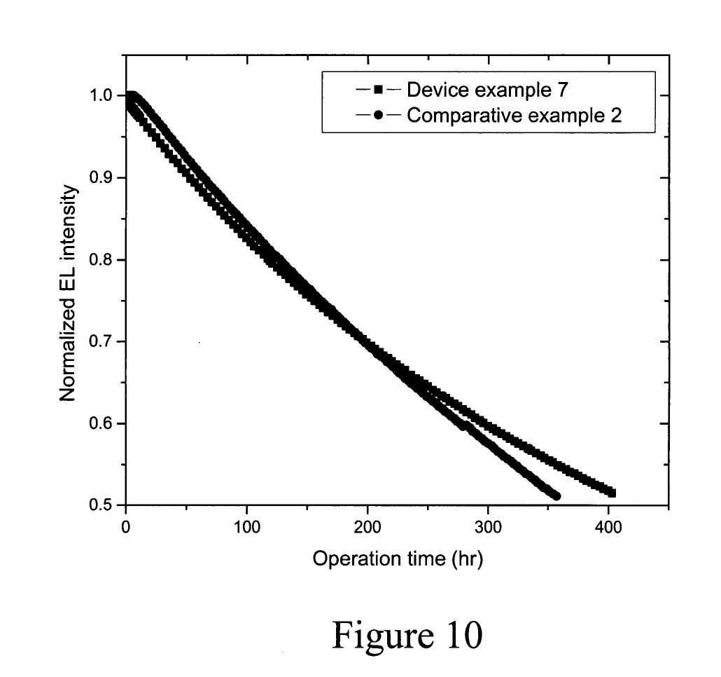

FIG. 5 shows plots of current density vs. voltage of device Example 7 and Comparative Example 2. FIG. 6 shows plots of external quantum efficiency vs. current density of device Example 7 and Comparative Example 2. FIG. 10 shows plots of operation lifetime of device Example 7 and Comparative Example 2.

### EXAMPLE 8

The organic stack consisted of sequentially, from the ITO surface, 100 Å thick of copper phthalocyanine (CuPc) as the hole injection layer (HIL), 400 Å of 4,4′-bis[N-(1-naphthyl)-N-phenylamino]biphenyl (α-NPD), as the hole transporting layer (HTL), 300 Å of 2,7-N,N-dicarbazolephenanthrene (2,7-DCP) doped with 12 wt % of Ir(1-piq)₃ as the emissive layer (EML), 100 Å of aluminum(III)bis(2-methyl-8-hydroxyquinolinato)4-phenylphenolate (BAlq) as the ETL2, and 500 Å of tris(8-hydroxyquinolinato)aluminum (Alq₃) as the ETL1.

### EXAMPLE 9

The organic stack consisted of sequentially, from the ITO surface, 100 Å thick of copperphthalocyanine (CuPc) as the hole injection layer (HIL), 400 Å of 4,4′-bis[N-(1-naphthyl)-N-phenylamino]biphenyl (α-NPD), as the hole transporting layer (HTL), 300 A of 2,7-N,N-dicarbazolephenanthrene (2,7-DCP) doped with 12 wt % of Ir(1-piq)₃ as the emissive layer (EML), 50 Å of 2,3,6,7,10,11-hexaphenyltriphenylene (HPT) as the ETL2, and 500 Å of tris(8-hydroxyquinolinato)aluminum (Alq₃) as the ETL1.

### EXAMPLE 10

The organic stack consisted of sequentially, from the ITO surface, 100 Å thick of copper phthalocyanine (CuPc) as the hole injection layer (HIL), 400 Å of 4,4′-bis[N-(1-naphthyl)-N-phenylamino]biphenyl (α-NPD), as the hole transporting layer (HTL), 300 Å of 2,7-N,N-dicarbazolephenanthrene (2,7-DCP) doped with 12 wt % of Ir(1-piq)₃ as the emissive layer (EML), and 500 Å of tris(8-hydroxyquinolinato)aluminum (Alq₃) as the ETL2. There was no ETL1.

### EXAMPLE 11

The organic stack consisted of sequentially, from the ITO surface, 100 Å thick of copper phthalocyanine (CuPc) as the hole injection layer (HIL), 400 Å of 4,4′-bis[N-(1-naphthyl)-N-phenylamino]biphenyl (α-NPD), as the hole transporting layer (HTL), 300 A of 2,7-N,N-dicarbazolephenanthrene (2,7-DCP) doped with 6 wt % of Ir(1-piq)₃ as the emissive layer (EML), and 50 Å of 2,3,6,7,10,11-hexaphenyltriphenylene (HPT) as the ETL2, and 500 Å of tris(8-hydroxyquinolinato)aluminum (Alq₃) as the ETL1.

### EXAMPLE 12

The organic stack consisted of sequentially, from the ITO surface, 100 Å thick of copper phthalocyanine (CuPc) as the hole injection layer (HIL), 400 Å of 4,4′-bis[N-(1-naphthyl)-N-phenylamino]biphenyl (α-NPD), as the hole transporting layer (HTL), 300 Å of 2,7-N,N-dicarbazolephenanthrene (2,7-DCP) doped with 8 wt % of Ir(1-piq)3 as the emissive layer (EML), and 50 Å of 2,3,6,7,10,11-hexaphenyltriphenylene (HPT) as the ETL2, and 500 Å of tris(8-hydroxyquinolinato)aluminum (Alq₃) as the ETL1.

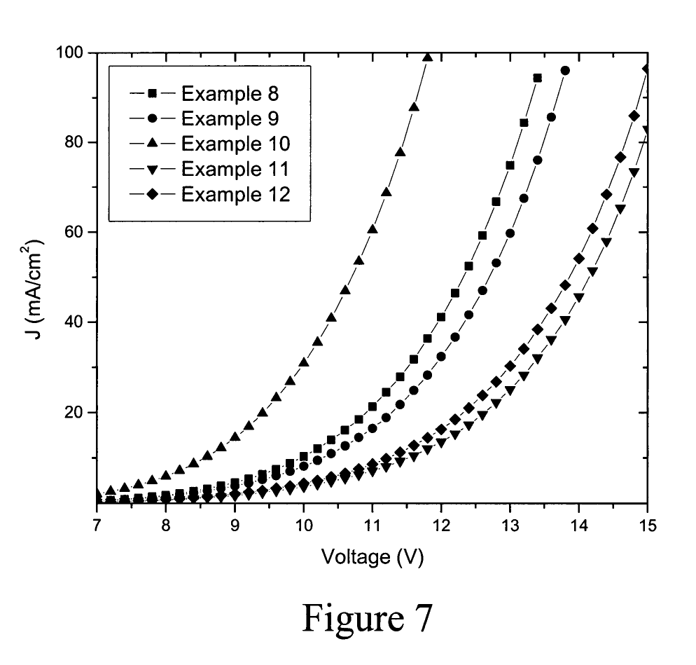

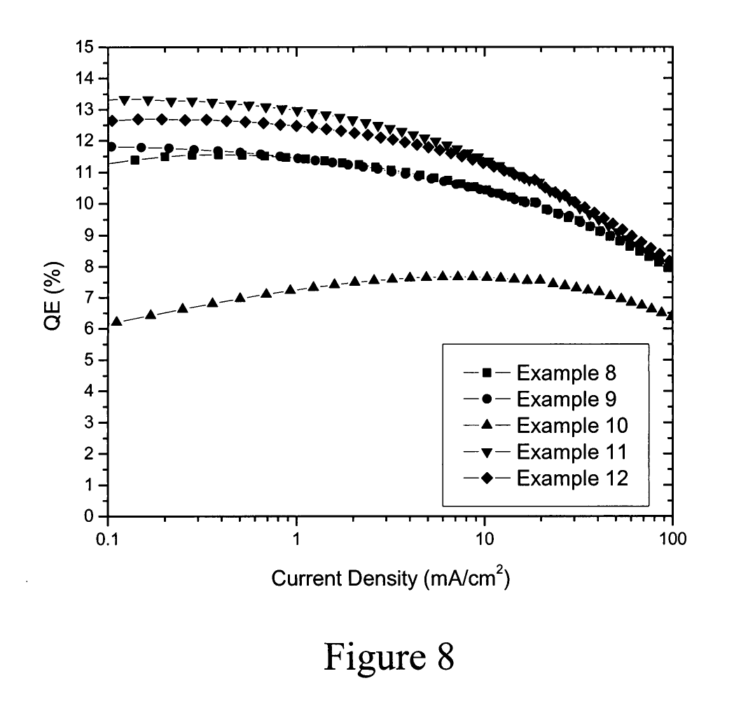

FIG. 7 shows plots of current density vs. voltage of device Examples 8, 9, 10, 11, and 12. FIG. 8 shows plots of external quantum efficiency vs. current density of device Examples 8, 9, 10, 11, and 12.

### EXAMPLE 13

The organic stack consisted of sequentially, from the ITO surface, 100 Å thick of copper phthalocyanine (CuPc) as the hole injection layer (HIL), 300 Å of 4,4′-bis[N-(1-naphthyl)-N-phenylamino]biphenyl (α-NPD), as the hole transporting layer (HTL), 300 Å of 3,3′-N,N-dicarbazole-o-terphenyl (3,3′-DC-o-TerP) doped with 6 wt % of Ir(5-Phppy)₃ as the emissive layer (EML), 50 Å of 2,3,6,7,10,11-hexaphenyltriphenylene (HPT) as the ETL2, and 450 Å of tris(8-hydroxyquinolinato)aluminum (Alq₃) as the ETL1.

### EXAMPLE 14

The organic stack consisted of sequentially, from the ITO surface, 100 Å thick of copper phthalocyanine (CuPc) as the hole injection layer (HIL), 300 Å of 4,4′-bis[N-(1-naphthyl)-N-phenylamino]biphenyl (α-NPD), as the hole transporting layer (HTL), 300 Å of 3,3′-N,N-dicarbazole-o-terphenyl (3,3′-DC-o-TerP) doped with 6 wt % of Ir(5-Phppy)₃ as the emissive layer (EML), 100 Å of aluminum(III)bis(2-methyl-8-hydroxyquinolinato)4-phenylphenolate (BAlq) as the ETL2, and 450 Å of tris(8-hydroxyquinolinato)aluminum (Alq₃) as the ETL1.

### EXAMPLE 15

The organic stack consisted of sequentially, from the ITO surface, 100 Å thick of copper phthalocyanine (CuPc) as the hole injection layer (HIL), 300 Å of 4,4′-bis[N-(1-naphthyl)-N-phenylamino]biphenyl (α-NPD), as the hole transporting layer (HTL), 300 Å of 3,3′-N,N-dicarbazole-o-terphenyl (3,3′-DC-o-TerP) doped with 6 wt % of Ir(5-Phppy)₃ as the emissive layer (EML), and 450 Å of tris(8-hydroxyquinolinato)aluminum (Alq₃) as the ETL2. There is no ETL1

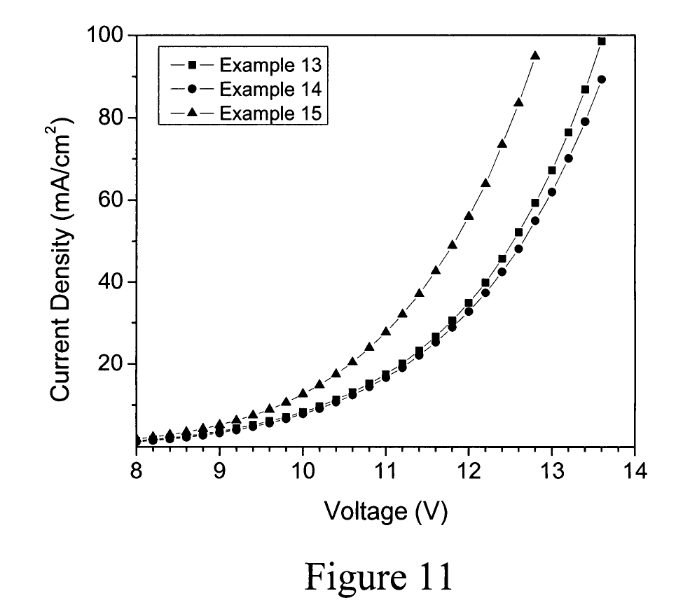

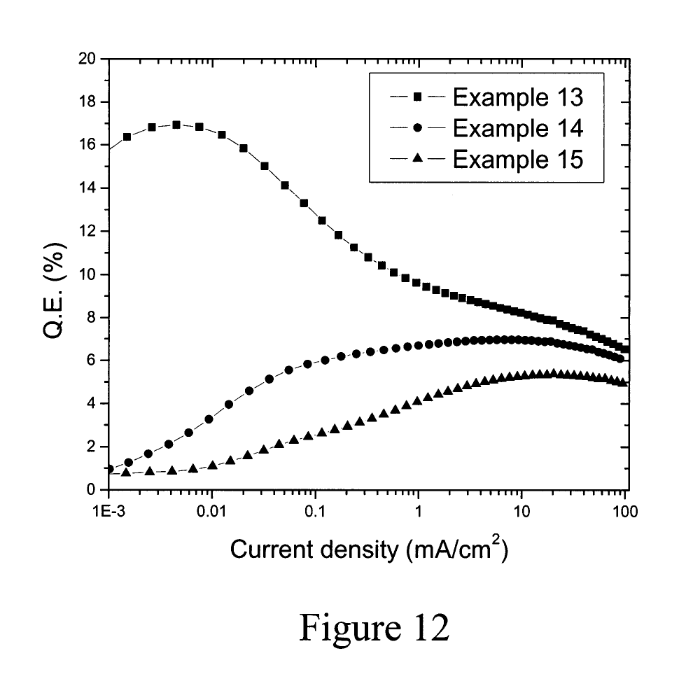

FIG. 11 shows plots of the current density vs. voltage of device Examples 13, 14 and 15. FIG. 12 shows plots of external quantum efficiency vs. current density of devices Examples 13, 14 and 15. FIG. 12 shows plots of operation lifetime of device Examples 13, 14 and 15.

While the present invention is described with respect to particular examples and preferred embodiments, it is understood that the present invention is not limited to these examples and embodiments. The present invention as claimed therefore includes variations from the particular examples and preferred embodiments described herein, as will be apparent to one of skill in the art.

What is claimed is:

1. A device, comprising:
an anode;
a cathode;
an emissive layer disposed between the anode and the cathode, wherein the emissive layer comprises a host and a dopant, and wherein the host material is selected from the group consisting of: 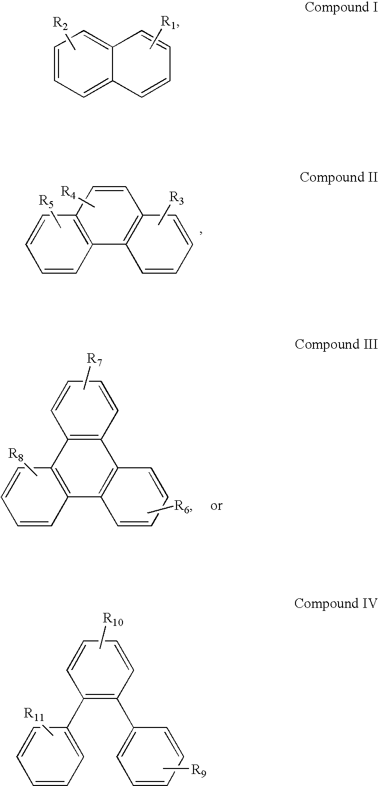
wherein each R represent no substitution, mono-, di-, or tri- substitution, and wherein the substituents are the same or different, and each is selected from the group consisting of alkyl, alkenyl, alkynyl, aryl, thioalkoxy, halo, haloalkyl, cyano, carbonyl, carboxyl, heteroaryl, and substituted aryl, and wherein at least one R for each Compounds I, II, III, or IV includes a carbazole group.

2. The device of claim 1, wherein each R represents no substitution, mono-, di-, or tri-substitution and all substituents are carbazole.

3. The device of claim 1, wherein the host material has the formula: 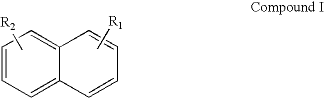
and wherein at least one of R₁ and R₂ includes a carbazole group.

4. The device of claim 3, wherein R₁ and R₂ each include a carbazole group.

5. The device of claim 3, wherein the host material has the formula:

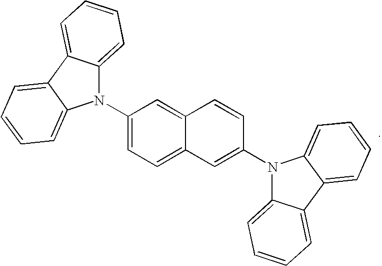

6. The device of claim 1, wherein the host material has the formula: 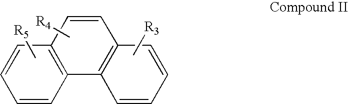
and wherein at least one of R₃, R₄, and R₅ includes a carbazole group.

7. The device of claim 6, wherein R₃ and R₅ each include a carbazole group.

8. The device of claim 6, wherein the host material has the formula:

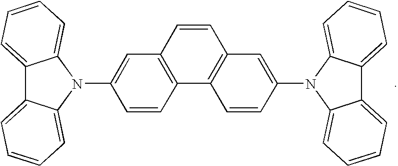

9. The device of claim 1, wherein the host material has the formula: 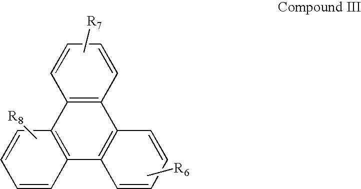
and wherein at least one of R₆, R₇, and R₈ includes a carbazole group.

10. The device of claim 9, wherein R₆ and R₈ each include a carbazole group.

11. The device of claim 9, wherein the host material has the formula:

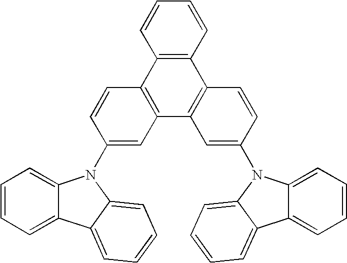

12. The device of claim 9, wherein the host material has the formula:

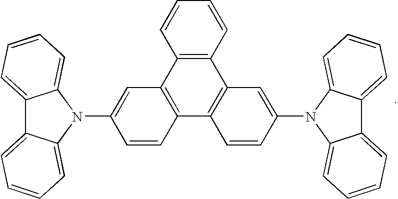

13. The device of claim 1, wherein the host material has the formula: 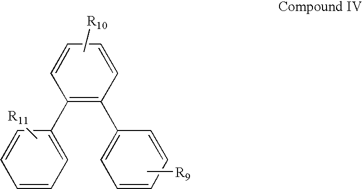
and wherein at least one of R₉, R₁₀, and R₁₁ includes a carbazole group.

14. The device of claim 13, wherein R₉ and R₁₁ each include a carbazole group.

15. The device of claim 13, wherein the host material has the formula:

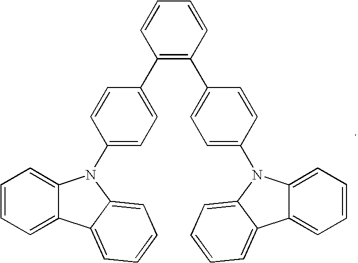

16. The device of claim 13, wherein the host material has the formula:

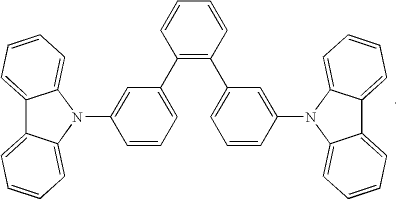

17. The device of claim 1, wherein the dopant is a phosphorescent emissive material.

18. A device, comprising:
an anode;
a cathode;
an organic layer disposed between the anode and the cathode, wherein the organic layer comprises a material selected from the group consisting of: 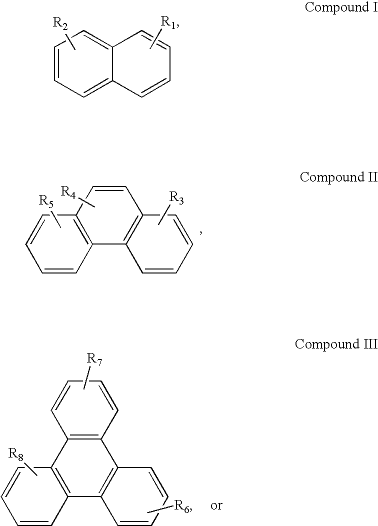 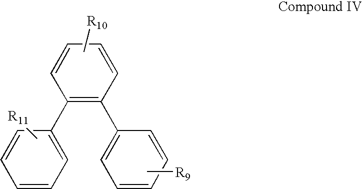
wherein each R represent no substitution, mono-, di-, or tri- substitution, and wherein the substituents are the same or different, and each is selected from the group consisting of alkyl, alkenyl, alkynyl, aryl, heteroaryl, thioalkoxy, halo, haloalkyl, cyano, carbonyl, carboxyl, and substituted aryl, and wherein at least one R for each Compounds I, II, III, or IV includes a carbazole group.

19. The device of claim 18, wherein each R represent no substitution, mono-, di-, or tri-substitution and is a carbazole group.

20. A compound, having a formula selected from the group consisting of: 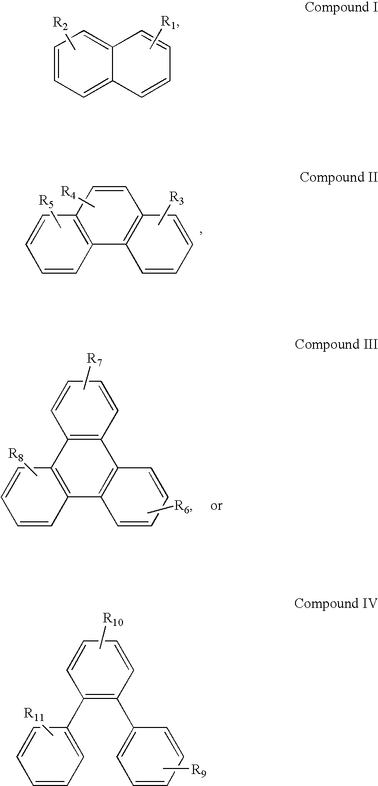
wherein each R represent no substitution, mono-, di-, or tri- substitution, and wherein the substituents are the same or different, and each is selected from the group consisting of alkyl, alkenyl, alkynyl, aryl, heteroaryl, thioalkoxy, halo, haloalkyl, cyano, carbonyl, carboxyl, and substituted aryl, and wherein at least one R for each Compounds I, II, III, or IV includes a carbazole group.

21. The compound of claim 20, wherein each R represents no substitution, mono-, di-, or tri-substitution and all substituents are carbazole.

22. The compound of claim 20, having the formula: 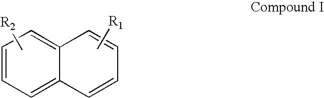
and wherein at least one of R₁ and R₂ includes a carbazole group.

23. The compound of claim 22, wherein R₁ and R₂ each include a carbazole group.

24. The compound of claim 22, having the formula:

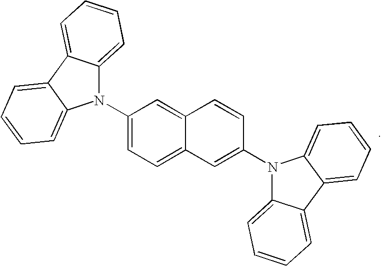

25. The compound of claim 20, having the formula: 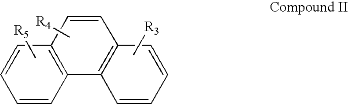
and wherein at least one of R₃, R₄, and R₅ includes a carbazole group.

26. The compound of claim 25, wherein R₃ and R₅ each include a carbazole group.

27. The compound of claim 25, having the formula:

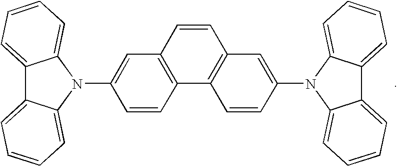

28. The compound of claim 20, having the formula: 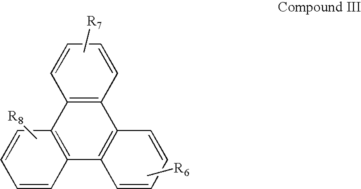
and wherein at least one of R₆, R₇, and R₈ includes a carbazole group.

29. The compound of claim 28, wherein R₆ and R₈ each include a carbazole group.

30. The compound of claim 28, having the formula:

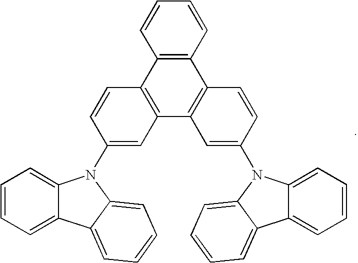

31. The compound of claim 28, having the formula:

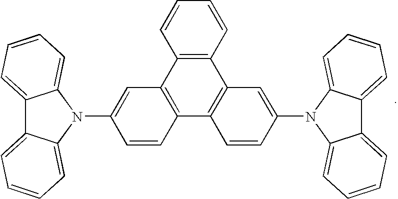

32. The compound of claim 20, having the formula: 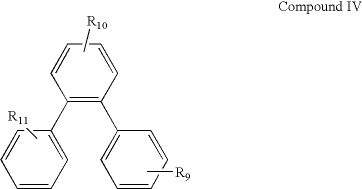
and wherein at least one of R₉, R₁₀, and R₁₁ includes a carbazole group.

33. The compound of claim 32, wherein R₉ and R₁₁ each include a carbazole group.

34. The compound of claim 32, having the formula:

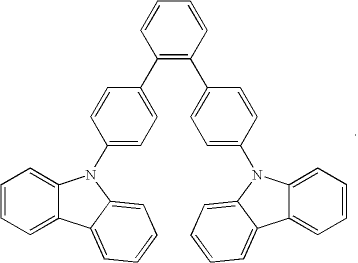

35. The compound of claim 32, having the formula: [chemical structure]
